(19) United States
(12) Patent Application Publication
(10) Pub. No.: US 2003/0181377 A1
(43) Pub. Date: Sep. 25, 2003

(54) Inhibition of VEGF receptor signaling reverses tumor resistance to radiotherapy

(75) Inventors: Dennis E. Hallahan, Nashville, TN (US); Charles Lin, Brentwood, TN (US)

Correspondence Address: JENKINS & WILSON, PA, 3100 TOWER BLVD, SUITE 1400, DURHAM, NC, 27707, US

(73) Assignee: Vanderbilt University

(21) Appl. No.: 10/364,949

(22) Filed: Feb. 12, 2003

Related U.S. Application Data
(60) Provisional application No. 60/356,309, filed on Feb. 12, 2002.

(51) Int. Cl. A61K 38/17; C07K 14/705

(52) U.S. Cl. 514/12; 530/350

(57) ABSTRACT

A method for increasing radiosensitivity of a tumor in a subject via administration of a VEGF-R2 inhibitor to a tumor in a subject. Also provided are methods for delaying tumor growth and for inhibiting tumor blood vessel growth via administration of a VEGF-R2 inhibitor.

### CROSS REFERENCE TO RELATED APPLICATIONS

This application is based on and claims priority to U.S. Provisional Application Serial No. 60/356,309, filed Feb. 12, 2002, herein incorporated by reference in its entirety.

### GRANT STATEMENT

This work was supported by grants CA58508, CA89674, CA89888, CA70937, and CA90949 from U.S. National Institute of Health. Thus, the U.S. government has certain rights in the invention.

### FIELD OF THE INVENTION

The present invention generally relates to compositions and methods for enhancing radiotherapy via inhibition of VEGF signaling. More particularly, the method involves administration of a VEGF-R2 inhibitor to a tumor in a subject, whereby the radiosensitivity of the tumor is increased.

Table of Abbreviations

| | |
|---|---|
| AAV | adeno-associated virus |
| Ad.ExFlk | adenovirus encoding soluble extracellular |
| | component of Flk-1 |
| Ad.LacZ | adenovirus encoding β-galactosidase |
| ATCC | American Type Culture Collection |
| B16F0 | murine melanoma cell line |
| CEA | carcinoembryonic antigen |
| D54 | human glioma cell line |
| Dil | 1,1′-dioctadecyl-3,3,3′,3′- |
| | tetramethylindocarbocyanine perchlorate |
| DMEM | Dulbecco's Modified Eagle Medium |
| EPCs | endothelial progenitor cells |
| ExFlk | soluble extracellular component of Flk-1 |
| ExFlk.6His | soluble extracellular component of Flk-1 |
| | tagged with 6 histidine residues |
| Flk-1 | fetal liver kinase 1; murine VEGF-R2 |
| Flt-1 | fms-like tyrosine kinase 1; VEGF-R1 |
| GBM | glioblastoma multiforme |
| GL261 | murine glioblastoma cell line |
| Gy | gray |
| HMEC | human microdermal endothelial cells |
| HSPs | high scoring sequence pairs |
| HUVEC | human umbilical vein endothelial cells |
| ¹²⁵I | iodine-125 |
| ¹³¹I | iodine-131 |
| IFN-α | interferon alpha |
| IFN-γ | interferon gamma |
| IL-2 | interleukin 2 |
| IL-4 | interleukin 4 |
| IL-6 | interleukin 6 |
| IMRT | intensity modulated radiation therapy |
| ITR | inverted terminal repeat |
| KDR | kinase insert domain receptor; human |
| | VEGF-R2 |
| Kᵢ | inhibition constant |
| LLC | Lewis lung carcinoma cell line |
| LTR | long terminal repeat |
| p | probability |
| ³²P | phosphorus-32 |
| PBS | phosphate buffered saline |
| PECAM | platelet-endothelial cell adhesion |
| | molecule-1/CD31 |
| pfu | plaque forming unit |
| SDS | sodium dodecyl sulfate |
| sFlk-1 | soluble extracellular component of Flk-1 |
| SSC | standard saline citrate |
| SU5416 | 3-[2, 4-dimethyl pyrrol-5-yl) methyl idenyl]- |
| | 2-indolinone |
| Tₘ | thermal melting point |
| TNF | tumor necrosis factor |
| VEGF | vascular endothelial growth factor |
| VEGF-R | vascular endothelial growth factor receptor |
| | (generally) |
| VEGF-R1 | vascular endothelial growth factor receptor |
| | type 1; |
| | Flt-1 |
| VEGF-R2 | vascular endothelial growth factor receptor |
| | type 2;Flk-1/KDR |

### BACKGROUND OF THE INVENTION

The inability of local radiation to control tumor growth is a significant clinical outcome leading to unsuccessful cancer therapy. See e.g., Suit, 1996 and Lindegaard et al., 1996. Tumors can be resistant to ionizing radiation, or tumors can recur because it is not possible to deliver a sufficiently high dose of radiation without an unacceptably high risk of damage to non-tumor cells.

One complication in designing radiotherapy is different cellular responses to ionizing radiation observed in vitro and in vivo. For example, glioblastoma multiforme (GBM) has a universally fatal clinical outcome in both children and adults (Walker et al., 1980; Wallner et al., 1989; Packer, 1999). In vitro studies show that numerous human GBM cell lines have radiosensitivity that is in the range of cell lines from more curable human tumors (Allam et al., 1993; Taghian et al., 1993). However, studies of the radiation response in GBM tumors in animal models have shown that the radioresistance of GBM tumors in vivo does not correlate with the radiosensitivity of the same cell lines in vitro (Baumann et al., 1992; Allam et al., 1993; Taghian et al., 1993; Advani et al., 1998; Staba et al., 1998).

Tumor cells could show radiosensitivity in vitro due to the absence of an angiogenic network that appears to contribute to radioresistance in vivo. The response of tumor microvasculature to radiation is dependent upon the dose and time interval after treatment (Kallman et al., 1972; Song et al., 1972; Hilmas & Gillette, 1975; Johnson, 1976; Yamaura et al., 1976; Ting et al., 1991). Tumor blood flow decreases when high doses in the range of 20 grays (Gy) to 45 Gy are used (Song et al., 1972). In contrast, blood volume increases when relatively low radiation doses, for example below 500 rads, are administered (Johnson, 1976). In irradiated mouse sarcomas, blood flow was observed to increase during 3 to 7 days following irradiation (Kallman et al., 1972; Hilmas & Gillette, 1975; Johnson, 1976; Yamaura et al., 1976; Gorski et al., 1999).

Increased tumor blood flow following radiation exposure could result from radiation-induced expression of angiogenic factors, such as vascular endothelial growth factor (VEGF) (Gorski et al., 1999). VEGF and vascular endothelial growth factor receptor type 2 (VEGF-R2; also called Flk-1 and KDR) expression levels increase in a time- and dose-dependent manner following irradiation of cultured human endothelial cells or tumor models (Gorski et al., 1999; Kermani et al., 2001). Endothelial cell proliferation and survival after in vitro irradiation are enhanced by supplementation of cell cultures with VEGF (Gorski et al., 1999), whereas anti-VEGF antibody enhances cytotoxic effects of irradiation. Binding of VEGF to the Flk-1/KDR receptor contributes to the VEGF-survival phenotype (Millauer et al., 1993; Waltenberger et al., 1994).

Levels of VEGF are also correlated with radiation stress and radiotherapy resistance (Shintani et al., 2000). VEGF is observed at increased levels in tumors that are resistant to radiotherapy such as malignant gliomas and melanomas. VEGF expression is also associated with worsened prognosis (Valter et al., 1999).

Despite these advances in understanding molecular and cellular bases for radioresistance, there exists a long-felt need in the art for effective therapies for promoting regression of tumors that are resistant to conventional therapies, including radiotherapy. To meet this need, the present invention provides in one embodiment a method for enhancing the radiosensitivity of a tumor via administration of a VEGF-R2 inhibitor. Also provided is a method for inhibiting tumor growth by: (a) administration of a VEGF-R2 inhibitor, whereby the tumor is radiosensitized; and (b) treating the tumor with ionizing radiation.

### SUMMARY OF THE INVENTION

In one embodiment, the present invention provides a method for increasing radiosensitivity of a tumor in a subject, wherein the method comprises administering a soluble VEGF-R polypeptide to a subject, such as by administering the soluble VEGF-R polypeptide to a tumor in a subject, whereby the radiosensitivity of the tumor is increased.

The present invention provides a method for increasing radiosensitivity of a tumor in a subject comprising administering a VEGF-R2 inhibitor to a tumor in a subject, whereby the radiosensitivity of the tumor is increased.

The present invention also provides a method for delaying tumor growth, the method comprising: (a) administering a VEGF-R2 inhibitor to a subject bearing a tumor; whereby the radiosensitivity of the tumor is increased; and (b) treating the tumor with ionizing radiation, whereby tumor growth is delayed. In one embodiment, the tumor growth delay comprises tumor regression.

The present invention also provides a method for inhibiting tumor blood vessel growth, the method comprising: (a) administering a VEGF-R2 inhibitor to a subject bearing a tumor; whereby the radiosensitivity of the tumor is increased; and (b) treating the tumor with ionizing radiation, whereby tumor blood vessel growth is inhibited. A VEGF-R2 inhibitor can also be administered after irradiation as maintenance therapy for the prevention of vascular regrowth.

The methods of the invention are useful for radiosensitizing tumors, delaying tumor growth, and inhibiting tumor vascularization in mammalian subjects. In one embodiment, a subject is a human subject. The methods are useful for treatment of all tumor types, and are particularly relevant for the treatment of radiation resistant tumors.

In one embodiment, a VEGF-R2 inhibitor comprises SU5416, a small molecule that specifically binds VEGF-R2. In another embodiment, a VEGF-R2 inhibitor comprises a soluble VEGF-R polypeptide.

A soluble VEGF-R polypeptide, including a soluble VEGF-R2 polypeptide and a soluble VEGF-R1 polypeptide, can comprise: (a) a polypeptide comprising an amino acid sequence of any one of even-numbered SEQ ID NOs: 2-8; (b) a polypeptide substantially identical to any one of even-numbered SEQ ID NOs: 2-8; (c) a polypeptide encoded by a nucleic acid of any one of odd-numbered SEQ ID NOs: 1-7; or (d) a polypeptide encoded by a nucleic acid substantially identical to any one of odd-numbered SEQ ID NOs: 1-7.

A soluble VEGF-R polypeptide can also comprise a polypeptide encoded by an isolated nucleic acid segment selected from the group consisting of: (a) an isolated nucleic acid molecule which hybridizes to a nucleic acid sequence of any one of odd-numbered SEQ ID NOs: 1-7 under wash stringency conditions represented by a wash solution having less than about 200 mM salt concentration and a wash temperature of greater than about 45° C., and which encodes a VEGF-R polypeptide; and (b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

A VEGF-R2 inhibitor is administered in one embodiment as a minimally therapeutic dose, although higher doses can be used as well. A subtherapeutic dose or a therapeutic dose of ionizing radiation can be used.

In accordance with the methods of the present invention, a VEGF-R2 inhibitor is provided via administration of a composition comprising: (a) a VEGF-R2 inhibitor; and (b) a pharmaceutically acceptable carrier. In one embodiment of the invention, the carrier comprises a gene therapy construct that encodes a soluble VEGF-R polypeptide.

Accordingly, it is an object of the present invention to provide novel methods for radiosensitizing a tumor to thereby delay tumor growth and to inhibit tumor vascularization. This and other objects are achieved in whole or in part by the present invention.

An object of the invention having been stated above, other objects and advantages of the present invention will become apparent to those of ordinary skill in the art after a study of the following description of the invention and non-limiting Examples.

### BRIEF DESCRIPTION OF THE DRAWINGS

FIG. 1 is a bar graph of the percent change in vascular length density of tumors treated with the radiation doses indicated. Vascular windows were prepared, and the length of tumor blood vessels photographed from each vascular window was determined as described in Example 2. The sum of changes in the quantity of blood vessels over time was compared to that observed at 0 hours. Bars represent the mean percent change in vascular length density and the standard error of the mean determined in 5 experiments. Solid bars, GL261 tumors; open bars, B16F0 tumors.

FIG. 2 is a bar graph that depicts the percent change in Power Doppler signal of tumors treated with the radiation doses indicated. GL261 and B16F0 tumor cell lines were pelleted and implanted into the hind limb of C57BL6 mice. Tumors were grown to diameters ranging from 0.7 cm to 1.1 cm and were irradiated. Tumor vasculature was analyzed by Power Doppler sonography as described in Example 3. Changes in Power Doppler signals were compared to signals recorded immediately prior to irradiation. Solid bars, GL261 tumors; open bars, B16F0 tumors.

FIGS. 3A and 3B are bar graphs that show an enhanced decrease in tumor vasculature following radiotherapy in combination with VEGF-R2 inhibition.

FIG. 3A is a bar graph that depicts a decreased percent change in vascular length density in response to adenovirus vector Ad.ExFlk followed by radiotherapy. GL261 and B16F0 tumors were implanted into a dorsal skin fold window, and tumor vasculature was allowed to develop over the course of 1 week. Blood vessels were photographed at the time of treatment (0 hour) and each day following treatment. Changes in treated blood vessels were compared to the same blood vessels prior to treatment (100%). The mean percent change in vascular length density and the standard error of the mean were determined at 72 hours following treatment. Additional methods are described in Example 5. Checkered bars, tumors treated with a control adenovirus vector encoding β-galactosidase (Ad.LacZ) followed by 2 Gy ionizing radiation 24 hours later; striped bars, tumors treated with 2 Gy ionizing radiation; open bars, tumors treated with an adenovirus vector encoding soluble Flk-1 (Ad.ExFlk); solid bars, tumors treated with 2 Gy ionizing radiation; open bars, tumors treated with an adenovirus vector encoding soluble Flk-1 (Ad.ExFlk) followed by 2 Gy ionizing radiation 24 hours later.

FIG. 3B is a bar graph that depicts a decreased percent change in vascular length density in response to SU5416 treatment followed by radiotherapy. GL261 and B16F0 tumors were implanted into a dorsal skin fold window, and tumor vasculature was allowed to develop over the course of 1 week. Blood vessels were photographed at the time of treatment (0 hour) and each day following treatment. Changes in treated blood vessels were compared to the same blood vessels prior to treatment (100%). Additional methods are described in Example 5. The mean percent change in vascular length density and the standard error of the mean were determined at 72 hours and 120 hours following treatment. Solid bars, B16F0 tumors; open bars, GL261 tumors.

FIG. 4 is a bar graph depicting a decreased percent change in Power Doppler signal in response to SU5416 followed by radiotherapy. GL261 and B16F0 tumors were implanted into the hind limb of C57BL6 mice. Tumors were grown to the diameter of approximately 1 cm and animals were treated by intraperitoneal administration of SU5416 with or without radiation with 3 Gy ionizing radiation fractions given on 8 days over the course of 11 days. Tumor vascularity was measured by Power Doppler analysis, and changes in tumor vascularity were determined relative to the same tumor at the time of treatment (0 hour, 100%). The mean percent change in Power Doppler signal and the standard error of the mean were determined using 3 mice in each treatment group. Additional methods are described in Example 5. Solid bars, B16F0 tumors; open bars, GL261 tumors.

FIG. 5 is a bar graph depicting an increased percentage of endothelial cells undergoing apoptosis in response to VEGF-R2 inhibition followed by radiotherapy. HUVEC cells (human umbilical vein endothelial cells), HMECs (human microdermal endothelial cells), and murine endothelial 3B11 cells were treated with 50 μM SU5416, 108 plaque-forming units (pfu) of Ad.ExFlk, or 10⁸ pfu control vector Ad.LacZ. Cells were treated. with 6 Gy of radiation and stained 24 hours later. The percentage of cells undergoing apoptosis was determined for each of the treatment groups as described in Example 6. The mean percentage of apoptotic cells and the standard error of the mean were determined using 4 culture plates in each treatment group.

FIGS. 6A and 6B are line graphs depicting an enhanced decrease in tumor volume in response to SU5416 followed by radiotherapy. GL261 cells were pelleted and implanted into the hind limb of C57BL6 mice. Tumors were grown to diameters ranging from 5 mm to 7 mm at which time treatment was initiated (day 0). Tumor volumes were measured at the indicated time points for each of the treatment groups. Mice were sacrificed when tumor volume exceeded 6 times the original tumor volume. Control mice received no therapy. SU5416 was administered by intraperitoneal injection twice per week for a total of 4 treatments on days 0, 4, 7 and 11. Ad.ExFlk and Ad.LacZ were administered by tail vein injection. Radiation was administered as 3 Gy fractions on 8 occasions on days 0, 1, 2 and 3. Additional methods are described in Example 7.

FIG. 6A is a line graph depicting the fractional change in tumor volume in response to the following treatments: (▪), untreated control; (▴), tumors treated with 3 Gy ionizing radiation; (♦), tumors treated with SU5416; and (●), tumors treated with SU5416 followed by 3 Gy ionizing radiation. The mean fractional change in tumor volume and the standard error of the mean were determined using 6 animals in each treatment group.

FIG. 6B is a line graph depicting the fractional change in tumor volume in response to the following treatments: (▪), untreated control; (▴), tumors treated with 3 Gy ionizing radiation; (♦), tumors treated with SU5416; and (●), tumors treated with SU5416 and 3 Gy ionizing radiation. The mean fractional change in tumor volume and the standard error of the mean were determined using 10 animals in each treatment group.

FIG. 7 is a bar graph depicting inhibition of tumor revascularization via administration of a VEGF-R2 inhibitor. Doppler analysis of blood flow in four tumor types is shown on 3, 5, and 7 days following radiation treatment. Animals received radiation treatment alone (control) or in combination with SU5416. In control animals, tumor revascularization occurs within 7 days following radiation treatment. Tumor revascularization is repressed in animals receiving SU5416 in combination with radiation treatment. Open bars, B16F0 tumors; solid bars, LLC tumors; stippled bars, GL261 tumors; hatched bars, D54 tumors.

### BRIEF DESCRIPTION OF THE SEQUENCE LISTING

SEQ ID NO: 1 is a nucleic acid sequence encoding a soluble Flk-1 receptor from mouse.

SEQ ID NO: 2 is the amino acid sequence of a polypeptide encoded by SEQ ID NO: 1.

SEQ ID NO: 3 is a nucleic acid sequence encoding a soluble human KDR polypeptide.

SEQ ID NO: 4 is the amino acid sequence of a polypeptide encoded by SEQ ID NO: 3.

SEQ ID NO: 5 is a nucleic acid sequence encoding a soluble mouse Flt-1 polypeptide.

SEQ ID NO: 6 is the amino acid sequence of a polypeptide encoded by SEQ ID NO: 5.

SEQ ID NO: 7 is a nucleic acid sequence encoding a soluble human Flt-1 polypeptide.

SEQ ID NO: 8 is the amino acid sequence of a polypeptide encoded by SEQ ID NO: 7.

### DETAILED DESCRIPTION OF THE INVENTION

I. Tumor Radiosensitivity

In view of the role of angiogenesis in promoting tumor recovery, cancer therapies have been developed that combine radiotherapy and anti-angiogenic agents. See Griscelli et al., 2000 and the references cited herein below. In particular, several studies have explored inhibition of the VEGF signaling pathway in combination with radiotherapy. In contrast to the studies cited herein below, the present invention provides a method for increasing the radiosensitivity of a tumor in a subject by administration of a VEGF-R2 inhibitor.

Two groups have described radiotherapy in combination with administration of an antibody that specifically binds VEGF, e.g. VEGF-A (Gorski et al., 1999; Lee et al., 2000). In contrast to the disclosure of the present invention, these studies did not demonstrate that inhibition of VEGF-R2 can increase tumor radiosensitivity.

In one embodiment, the present invention provides a novel method for increasing the radiosensitivity of a tumor in a subject via administration of a VEGF-R2 inhibitor. The present invention also provides a method for delaying tumor growth in a subject. The method comprises: (a) administering a VEGF-R2 inhibitor to a tumor in a subject, whereby the radiosensitivity of the tumor is increased; and (b) treating the tumor with ionizing radiation, whereby tumor growth is delayed and tumor regression is promoted and sustained.

As used herein, the term “VEGF-R1” refers to the Flt-1 receptor. The terms “VEGF-R1” and “Flt-1” are used interchangeably.

As used herein, the term VEGF-R2 refers to the Flk-1 receptor, which in humans is also called KDR. The terms “VEGF-R2”, “Flk-1”, and “KDR” are used interchangeably.

The term “radiosensitivity”, as used herein to describe a tumor, refers to a quality of susceptibility to treatment using ionizing radiation. Thus, radiotherapy can be used to delay growth of a radiosensitive tumor. Radiosensitivity can be quantified by determining a minimal amount of ionizing radiation that can be used to delay tumor growth. Thus, the term “radiosensitivity” refers to a quantitative range of radiation susceptibility.

The term “delaying tumor growth” refers to a decrease in a duration of time required for a tumor to grow a specified amount. For example, treatment can delay the time required for a tumor to increase in volume 3-fold relative to an initial day of measurement (day 0) or the time required to grow to 1 cm³.

The terms “radiation resistant tumor” and “radioresistant tumor” each generally refer to a tumor that is substantially unresponsive to radiotherapy when compared to other tumors. Representative radiation resistant tumor models include glioblastoma multiforme and melanoma.

The term “increase,” as used herein to refer to a change in radiosensitivity of a tumor, refers to change that renders a tumor more susceptible to destruction by ionizing radiation. Alternatively stated, an increase in radiosensitivity refers to a decrease in the minimal amount of ionizing radiation that effectively delays tumor growth. An increase in radiosensitivity can also comprise delayed tumor growth when a VEGF-R2 inhibitor is administered with radiation as compared to a same dose of radiation alone. In one embodiment, an increase in radiosensitvity refers to an increase of at least about 2-fold, in another embodiment an increase of at least about 5-fold, and in still another embodiment an increase of at least 10-fold. In yet another embodiment of the invention, an increase in radiosensitivity comprises a transformation of a radioresistant tumor to a radiosensitive tumor.

The term “tumor regression” generally refers to any one of a number of indices that suggest change within the tumor to a less developed form. Such indices include, but are not limited to a destruction of tumor vasculature (for example, a decrease in vascular length density or a decrease in blood flow), a decrease in tumor cell survival, a decrease in tumor volume, and/or a decrease in tumor growth rate. Representative methods for assessing tumor growth delay and tumor regression are described in Examples 2, 3, 6, and 7.

The methods of the present invention are useful for increasing the radiosensitivity of a tumor, for delaying tumor growth, and/or for inhibiting tumor vascularization in any tumor-bearing subject. Thus, the term “subject” as used herein includes any vertebrate species, in one embodiment warm-blooded vertebrates such as mammals and birds. More particularly, the methods of the present invention are contemplated for the treatment of tumors in mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economical importance to humans.

The term “tumor” as used herein encompasses both primary and metastasized solid tumors and carcinomas of any tissue in a subject, including, but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. The term “tumor” also encompasses radioresistant tumors, including, but not limited to radioresistant variants of the any of the tumor listed above.

The term “about”, as used herein when referring to a measurable value such as an amount of weight, time, dose (e.g. radiation dose), etc. is meant to encompass variations of in one embodiment ±20% or ±10%, in another embodiment ±5%, in another embodiment ±1%, and in still another embodiment ±0.1% from the specified amount, as such variations are appropriate to perform the disclosed method.

II. VEGF-R2 Inhibitors

The present invention provides methods for increasing radiosensitivity of a tumor via administration of a VEGF-R2 inhibitor. Any suitable VEGF-R2 inhibitor can be used in accordance with the methods of the present invention, wherein the inhibitor has a capacity to increase the radiosensitivity of a tumor. In one embodiment, a VEGF-R2 inhibitor also shows anti-angiogenic activity or angiostatic activity.

The term “VEGF-R2 inhibitor” as used herein refers to a molecule or other chemical entity having a capacity for specifically binding to VEGF-R2 to thereby inhibit a VEGF-R2 biological activity. VEGF-R2 inhibitors include, but are not limited to small molecule inhibitors, soluble VEGF-R polypeptides (i.e., soluble VEGF-R1 and soluble VEGF-R2), and antibodies that specifically bind VEGF-R2. In one embodiment, the term “VEGF-R2 inhibitor” excludes molecules, other than a soluble VEGF-R polypeptide, that specifically bind to VEGF, such as an anti-VEGF antibody.

In accordance with the present invention, however, a soluble VEGF-R polypeptide can specifically bind VEGF. As such, a soluble VEGF-R polypeptide of the present invention can specifically bind one or more of several different isoforms of VEGF including, but not limited to VEGF-A, VEGF-B, VEGF-C, VEGF-D, and VEGF-E. While applicants do not wish to be bound by any particular theory of operation, a VEGF-R2 inhibitor, such as a soluble VEGF-R polypeptide, could also function as an inhibitor of VEGF-R1 signaling as well by specifically binding one or more of the VEGF isoforms. Thus, for example, as a result of cross-reactivity between the soluble VEGF-R polypeptide and the various isoforms of VEGF, therefore, in certain embodiments the term “VEGF-R2 inhibitor” can encompass molecules that inhibit signaling through the VEGF-R2 receptor, even if the VEGF-R2 inhibitor also inhibits signaling through the VEGF-R1 receptor at the same time.

The term “binding” refers to an affinity between two molecules, for example, an inhibitor and a target molecule. As used herein, “specific binding” means a preferential binding of one molecule for another in a mixture of molecules. The binding of an inhibitor to a target molecule can be considered specific if the binding affinity is about 1×10⁴ M⁻¹ to about 1×10⁶ M⁻¹ or greater.

II.A. Small Molecules

The term “small molecule” as used herein refers to a compound, for example an organic compound, with a molecular weight of in one embodiment less than about 1,000 daltons, in another embodiment less than about 750 daltons, in another embodiment less than about 600 daltons, and in still another embodiment less than about 500 daltons. A small molecule also has a computed log octanol-water partition coefficient in the range of about −4 to about +14 in one embodiment, and in another embodiment in the range of about −2 to about +7.5.

SU5416 is a small molecule inhibitor of Flk-1/KDR that inhibits tyrosine kinase catalysis, tumor vascularization, and growth of multiple tumor types (Fong et al., 1999; Shaheen et al., 1999; Vajkoczy et al., 1999). U.S. Pat. No. 6,159,443 to Hallahan describes SU5416 as a radiosensitizing agent that becomes more toxic to a cell following exposure of the cell to ionizing radiation.

II.B. Soluble VEGF-R Polypeptides

The term “soluble VEGF-R polypeptide” refers to a VEGF-R polypeptide that inhibits VEGF-R2 signaling. As described above, a soluble VEGF-R polypeptide might also inhibit VEGF-R1 signaling. The soluble VEGF-R polypeptide comprises in one embodiment a truncated VEGF-R polypeptide having an ability to bind VEGF, to heterodimerize with native VEGF-R2, or a combination thereof. The truncated soluble forms of Flk-1 (VEGF-R2) and Flt-1 (VEGF-R1) inhibit VEGF signaling and delay tumor growth. Soluble Flk-1 and Flt-1 bind to and sequester VEGF, thereby limiting the availability of VEGF for binding to native Flk-1 (Leunig et al., 1992; Goldman et al., 1998; Lin et al, 1998). Soluble Flk-1 and soluble Flt-1 also function as dominant negative receptors by forming inactive heterodimers with native Flk-1 (Kendall et al., 1994; Kendall et al., 1996).

In one embodiment of the invention, a soluble VEGF-R polypeptide comprises soluble Flk-1 polypeptide (VEGF-R2) comprising: (a) a polypeptide encoded by a nucleic acid of SEQ ID NO: 1 or 3; (b) a polypeptide encoded by a nucleic acid substantially identical to SEQ ID NO: 1 or 3; (c) a polypeptide comprising an amino acid sequence of SEQ ID NO: 2 or 4; or (d) a polypeptide substantially identical to SEQ ID NO: 2 or 4.

In another embodiment of the invention, a soluble VEGF-R polypeptide comprises soluble Flt-1 polypeptide (VEGF-R1) comprising: (a) a polypeptide encoded by a nucleic acid of SEQ ID NO: 5 or 7; (b) a polypeptide encoded by a nucleic acid substantially identical to SEQ ID NO: 5 or 7; (c) a polypeptide comprising an amino acid sequence of SEQ ID NO: 6 or 8; or (d) a polypeptide substantially identical to SEQ ID NO: 6 or 8.

Nucleic Acids. The terms “nucleic acid molecule” and “nucleic acid” each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, or “mRNA”. Nucleic acids can be synthesized, or can be derived from any biological source, including any organism.

The term “substantially identical”, as used herein to describe a degree of similarity between nucleotide sequences, refers to two or more sequences that have in one embodiment at least about least 60%, in another embodiment at least about 70%, in another embodiment at least about 80%, in another embodiment about 90% to about 99%, in another embodiment about 95% to about 99%, and in still another embodiment about 99% nucleotide identity, when compared and aligned for maximum correspondence, as measured using a sequence comparison algorithm (described herein below under the heading “Nucleotide and Amino Acid Sequence Comparisons”) or by visual inspection. The substantial identity exists in one embodiment in nucleotide sequences of at least about 100 residues, in another embodiment in nucleotide sequences of at least about 150 residues, and in yet another embodiment in nucleotide sequences comprising a full length coding sequence. The term “full length”, as used herein refers to a complete open reading frame encoding a functional soluble VEGF-R polypeptide. Representative full-length nucleic acids encoding a soluble VEGF-R polypeptide are set forth as any one of odd-numbered SEQ ID NOs: 1-7. Full-length nucleic acids encoding a soluble VEGF-R2 polypeptide include, but are not limited to those set forth as SEQ ID NOs: 1 and 3.

In one aspect, substantially identical sequences can comprise polymorphic sequences. The term “polymorphic” refers to the occurrence of two or more genetically determined alternative sequences or alleles in a population. An allelic difference can be as small as one base pair.

In another aspect, substantially identical sequences can comprise mutagenized sequences, including sequences comprising silent mutations. A mutation can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

A nucleotide sequence employed for hybridization studies or assays includes probe sequences that in one embodiment are complementary to or mimic at least an about 14 to 40 nucleotide sequence of a nucleic acid molecule of the present invention. In another embodiment, probes comprise 14 to 20 nucleotides, or even longer where desired, such as 30, 40, 50, 60, 100, 200, 300, or 500 nucleotides or up to the full length of any one of odd-numbered SEQ ID NOs: 1-7. Such probes can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent hybridization and wash conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× SSC at 65° C. An example of stringent wash conditions is 15 minutes in 0.2× SSC buffer at 65° C. See Sambrook & Russell, 2001 for a description of SSC buffer. Often, a high stringency wash is preceded by a low stringency wash to remove background probe signal. An example of medium stringency wash conditions for a duplex of more than about 100 nucleotides is 15 minutes in 1× SSC at 45° C. An example of low stringency wash for a duplex of more than about 100 nucleotides is 15 minutes in 4× to 6× SSC at 40° C. For short probes (e.g., about 10 to 50 nucleotides), stringent conditions typically involve salt concentrations of less than about 1 M Na⁺ ion, typically about 0.01 to 1 M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the present invention: a probe nucleotide sequence hybridizes to a target nucleotide sequence in one embodiment in 7% sodium dodecyl sulfate (SDS), 0.5 M NaPO₄, 1 mM EDTA at 50° C. followed by washing in 2× SSC, 0.1% SDS at 50° C; in another embodiment, a probe and target sequence hybridize in 7% SDS, 0.5 M NaPO₄, 1 mM EDTA at 50° C. followed by washing in 1× SSC, 0.1% SDS at 50° C.; in another embodiment, a probe and target sequence hybridize in 7% SDS, 0.5 M NaPO₄, 1 mM EDTA at 50° C. followed by washing in 0.5× SSC, 0.1% SDS at 50° C.; in another embodiment, a probe and target sequence hybridize in 7% SDS, 0.5 M NaPO₄, 1 mM EDTA at 50° C. followed by washing in 0.1× SSC, 0.1% SDS at 50° C.; in still another embodiment, a probe and target sequence hybridize in 7% SDS, 0.5 M NaPO₄, 1 mM EDTA at 50° C. followed by washing in 0.1× SSC, 0.1% SDS at 65° C.

A further indication that two nucleic acid sequences are substantially identical is that the proteins encoded by the nucleic acids are substantially identical, share an overall three-dimensional structure, or are biologically functional equivalents. These terms are defined further under the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al., 1991; Ohtsuka et al., 1985; and Rossolini et al., 1994.

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising in one embodiment about 8 or more deoxyribonucleotides or ribonucleotides, in another embodiment 10-20 nucleotides, and in still another embodiment 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to a sequence comprising additional nucleotides (or other analogous molecules) incorporated into and/or at either end of a nucleic acid. For example, a polymerase (e.g., a DNA polymerase) can add sequences at the 3′ terminus of a nucleic acid molecule. In addition, a nucleotide sequence can be combined with other DNA sequences, including, but not limited to promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. An example of a complementary nucleic acid segment is an antisense oligonucleotide.

The term “gene” refers broadly to any segment of DNA associated with a biological function. A gene encompasses sequences including, but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including, but not limited to cloning from a biological sample, synthesis based on known or predicted sequence information, and recombinant derivation of an existing sequence.

The present invention also encompasses chimeric genes comprising a nucleotide sequence encoding a soluble VEGF-R polypeptide. The term “chimeric gene”, as used herein, refers to a gene comprising a heterologous promoter region operatively linked to a nucleotide sequence encoding a soluble VEGF-R polypeptide.

The term “operatively linked”, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term “heterologous,” as used herein to refer to a promoter or any other nucleic acid, refers to a sequence that originates from a source foreign to an intended host cell or, if from the same source, is modified from its original form. Thus, a heterologous nucleic acid in a host cell includes, but is not limited to a gene that is endogenous to the particular host cell but has been modified, for example by mutagenesis or by isolation from native cis-regulatory sequences. The term “heterologous nucleic acid” also includes non-naturally occurring multiple copies of a native nucleotide sequence. The term “heterologous nucleic acid” also encompasses a nucleic acid that is incorporated into a host cell's nucleic acids, however at a position wherein such nucleic acids are not ordinarily found. A representative heterologous nucleic acid comprises a recombinant nucleic acid, as described further herein below.

The term “recombinant” generally refers to an isolated nucleic acid that is replicable in a non-native environment. Thus, a recombinant nucleic acid can comprise a non-replicable nucleic acid in combination with additional nucleic acids, for example vector nucleic acids, which enable its replication in a host cell.

The term “vector” is used herein to refer to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors include plasmids, cosmids, and viral vectors. A vector can also mediate recombinant production of a soluble VEGF-R polypeptide.

The term “construct”, as used herein to describe an expression construct, refers to a vector further comprising a nucleotide sequence operatively inserted with the vector, such that the nucleotide sequence is expressed.

The terms “recombinantly expressed” or “recombinantly produced” are used interchangeably to generally refer to the process by which a polypeptide encoded by a recombinant nucleic acid is produced.

The term “heterologous expression system” refers to a host cell comprising a heterologous nucleic acid and the polypeptide encoded by the heterologous nucleic acid. For example, a heterologous expression system can comprise a host cell transfected with a construct comprising a recombinant nucleic acid, or a cell line produced by introduction of heteroliogous nucleic acids into a host cell genome.

Nucleic acids of the present invention can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art as exemplified by publications. See e.g., Sambrook & Russell, 2001; Silhavy et al., 1984; Glover & Hames, 1995; and Ausubel, 1995.

Polypeptides. In one embodiment, the present invention provides an viral construct encoding soluble VEGF-R polypeptides. The present invention also provides a recombinantly expressed and isolated soluble VEGF-R polypeptide. A representative soluble VEGF-R polypeptide comprises multiple immunoglobulin loops, but lacks a transmembrane domain and a kinase domain as present in native VEGF-R. Representative soluble VEGF-R polypeptides are set forth as any one of even-numbered SEQ ID NOs: 2-8. A soluble VEGF-R2 polypeptides is set forth in one embodiment as SEQ ID NO: 2 and in another embodiment as SEQ ID NO: 4.

The term “substantially identical”, as used herein to describe a level of similarity between a soluble VEGF-R polypeptide and a protein substantially identical to a soluble VEGF-R polypeptide, refers to a sequence that is at least 35% identical to any one of even-numbered SEQ ID NOs: 2-8, when compared over the full length of a soluble VEGF-R protein. A protein substantially identical to soluble VEGF-R comprises an amino acid sequence that is in one embodiment at least about 35% to about 45% identical to any one of even-numbered SEQ ID NOs: 2-8, in another embodiment at least about 45% to about 55% identical to any one of even-numbered SEQ ID NOs: 2-8, and in still another embodiment at least about 55% to about 65% identical to any one of even-numbered SEQ ID NOs: 2-8 when compared over the full length of a soluble VEGF-R polypeptide. Methods for determining percent identity are defined herein below under the heading “Nucleotide and Amino Acid Sequence Comparisons.”

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Saqi et al., 1999; Barton, 1998; Henikoff et al., 2000; and Huang et al., 2000.

Substantially identical proteins also include proteins comprising amino acids that are functionally equivalent to amino acids of any one of even-numbered SEQ ID NOs: 2-8. The term “functionally equivalent” in the context of amino acid sequences is known in the art and is based on the relative similarity of the amino acid side-chain substituents. See Henikoff & Henikoff, 2000. Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (-0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. In making changes based upon the hydropathic index, amino acids whose hydropathic indices are within NO: ±2 of the original value are substituted in one embodiment, those within ±1 of the original value are substituted in another embodiment, and those within NO: ±0.5 of the original value are substituted in yet another embodiment.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, amino acids whose hydrophilicity values are within ±2 of the original value are substituted in one embodiment, those which are within ±1 of the original value are substituted in another embodiment, and those within ±0.5 of the original value are substituted in yet another embodiment.

The term “substantially identical” also encompasses polypeptides that are biologically functional equivalents. The term “functional” includes activity of a soluble VEGF-R polypeptide in inhibiting VEGF-R2 signaling and increasing radiosensitivity of a tumor, as described herein. Methods for assessing a radiosensitizing function are described in the Examples.

The present invention also provides functional fragments of a soluble VEGF-R polypeptide. Such functional portion need not comprise all or substantially all of the amino acid sequence of an extracellular portion of a native VEGF-R gene product.

The present invention also includes functional polypeptide sequences that are longer sequences than that of an extracellular portion of a native soluble VEGF-R polypeptide. For example, one or more amino acids can be added to the N-terminus or C-terminus of a soluble VEGF-R polypeptide. Methods of preparing such proteins are known in the art.

Nucleotide and Amino Acid Sequence Comparisons. The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman, 1981, by the homology alignment algorithm of Needleman & Wunsch, 1970, by the search for similarity method of Pearson & Lipman, 1988, by computerized implementations of these algorithms (GAP, BESTFIT, FASTA, and TFASTA, in the Wisconsin Genetics Software Package, available from Accelrys Inc., San Diego, Calif., United States of America), or by visual inspection. See generally, Ausubel, 1995.

In one embodiment, an algorithm for determining percent sequence identity and sequence similarity is the BLAST algorithm, which is described by Altschul et al., 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

II.C. VEGF-R2 Antibodies

The present invention further provides a VEGF-R2 inhibitor comprising: (a) an antibody that specifically binds VEGF-R2; and (b) a carrier for sustained bioavailability of the antibody at a tumor. The disclosure herein reveals that a prolonged or sustained release of VEGF-R2 antagonist is required to optimize the therapeutic effect of combined VEGF-R2 antagonism and radiation.

The term “sustained bioavailability” is used herein to describe a composition comprising a VEGF-R2 inhibitor and a carrier, whereby the bioavailability of a VEGF-R2 inhibitor at a tumor site is sufficient to achieve radiosensitization of a tumor. The term “sustained bioavailability” also refers to a bioavailability sufficient to inhibit blood vessel growth within the tumor.

The term “sustained bioavailability” encompasses factors including but not limited to sustained release of a VEGF-R2 inhibitor from a carrier, metabolic stability of a VEGF-R2 inhibitor, systemic transport of a composition comprising a VEGF-R2 inhibitor, and effective dose of a VEGF-R2 inhibitor. Representative approaches for preparing a sustained bioavailability composition are described herein below under the heading “Carriers.”

Kozin et al., 2001 describes a decrease in the dose of radiation required to locally control 50% of human tumor xenografts by combination of radiotherapy with administration of an anti-VEGF-R2 antibody over a period of 6 months. In two tumor models, the combined effect of ionizing radiation and anti-VEGF-R2 antibody was not significantly different from additive. These studies did not include a carrier for sustained bioavailability of a VEGF-R2 antibody at the tumor site, and provision of the antibody failed to confer an increase in radiosensitivity of the tumor. In contrast, the present invention provides that a VEGF-R2 inhibitor comprising a VEGF-R2 antibody further comprises a carrier for sustained bioavailability. In one embodiment, a carrier for sustained bioavailability comprises a gene therapy construct encoding a VEGF-R2 antibody.

As disclosed herein, an immediate response of tumor blood vessels to radiation is a decrease in tumor blood flow. This response can diminish administration of an anti-tumor composition (e.g., a VEGF-R2 inhibitor). Recognizing this response, the disclosure of the present invention provides that sustained bioavailability of a VEGF-R2 inhibitor, for example by selection of a carrier and administration regimen that achieve sustained bioavailability, can improve anti-tumor activity. In one embodiment, a carrier comprises a gene therapy vector encoding a VEGF-R2 inhibitor.

A method comprising a carrier or administration approach for sustained bioavailability can also improve therapies directed toward modulation of other components of the VEGF signaling pathway. For example, treatments that employed direct administration of an anti-VEGF antibody (in the absence of a carrier or administration regimen for sustained bioavailability of the antibody at the tumor) showed variable anti-tumor efficacy in different tumor types (Gorski et al., 1999; Lee et al., 2000). In addition, therapeutic effects were not observed when sub-therapeutic or minimally therapeutic doses of the anti-VEGF antibody were used. Thus, the present invention further provides an improved method for inhibiting tumor growth, the method comprising administration of a gene therapy vector encoding an inhibitor of VEGF signaling, whereby bioavailability of the inhibitor at a tumor is sustained, and whereby tumor growth delay is improved.

The term “antibody” indicates an immunoglobulin protein, or functional portion thereof, including, but not limited to a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). In one embodiment, an antibody of the invention is a monoclonal antibody.

Techniques for preparing and characterizing antibodies are known in the art. See e.g., Harlow & Lane, 1988 and U.S. Pat. Nos. 4,196,265; 4,946,778; 5,091,513; 5,132,405; 5,260,203; 5,677,427; 5,892,019; 5,985,279; 6,054,561). Single chain antibodies can be identified by screening a phage antibody library, for example as described by U.S. Pat. Nos. 6,174,708; 6,057,098; 5,922,254; 5,840,479; 5,780,225; 5,702,892; and 5,667,988.

An antibody of the invention can further be mutagenized or otherwise modified to preferably improve antigen binding and/or antibody stability. For example, to prevent undesirable disulfide bond formation, a nucleotide sequence encoding the variable domain of an antibody or antibody fragment can be modified to eliminate at least one of each pair of codons that encode cysteines for disulfide bond formation. Recombinant expression of the modified nucleotide sequence, for example in a prokaryotic expression system, results in an antibody having improved stability. See U.S. Pat. No. 5,854,027.

III. Therapeutic Compositions

In accordance with the methods of the present invention, a composition that is administered to increase the radiosensitivity of a tumor in a subject comprises: (a) a VEGF-R2 inhibitor; and (b) a pharmaceutically acceptable carrier. Any suitable carrier that facilitates drug preparation and/or administration can be used.

III.A. Carriers

The carrier can be a viral vector or a non-viral vector. Suitable viral vectors include, but are not limited to adenoviruses, adeno-associated viruses (AAVs), retroviruses, pseudotyped retroviruses, herpes viruses, vaccinia viruses, Semiliki forest virus, and baculoviruses. In one embodiment of the invention, the carrier comprises an adenoviral gene therapy construct that encodes a VEGF-R2 inhibitor.

Suitable non-viral vectors that can be used to deliver a VEGF-R2 inhibitor include, but are not limited to a plasmid, a nanosphere (Manome et al., 1994; Saltzman & Fung, 1997), a peptide (U.S. Pat. Nos. 6,127,339 and 5,574,172), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al., 1997 and U.S. Pat. Nos. 4,551,482, 5,714,166, 5,510,103, 5,490,840, and 5,855,900), and a polysome (U.S. Pat. No. 5,922,545).

Where appropriate, two or more types of carriers can be used together. For example, a plasmid vector can be used in conjunction with liposomes. One embodiment of the present invention envisions the use of an adenovirus.

In one embodiment, a composition of the invention comprises a VEGF-R2 inhibitor and a carrier to effect sustained bioavailability of the VEGF-R2 inhibitor following administration to a tumor-bearing subject. The term “sustained bioavailability” is used herein to refer to a bioavailability of a VEGF-R2 inhibitor sufficient to achieve radiosensitization of a tumor. The term “sustained bioavailability” also refers to a bioavailability of a VEGF-R2 inhibitor sufficient to inhibit blood vessel growth within a tumor. The term “sustained bioavailability” encompasses factors including, but not limited to prolonged release of a VEGF-R2 inhibitor from a carrier, metabolic stability of a VEGF-R2 inhibitor, systemic transport of a composition comprising a VEGF-R2 inhibitor, and effective dose of a VEGF-R2 inhibitor.

Representative compositions for sustained bioavailability of a VEGF-R2 inhibitor include, but are not limited to polymer matrices, including swelling and biodegradable polymer matrices, (U.S. Pat. Nos. 6,335,035; 6,312,713; 6,296,842; 6,287,587; 6,267,981; 6,262,127; and 6,221,958), polymer-coated microparticles (U.S. Pat. Nos. 6,120,787 and 6,090,925) a polyol:oil suspension (U.S. Pat. No. 6,245,740), porous particles (U.S. Pat. No. 6,238,705), latex/wax coated granules (U.S. Pat. No. 6,238,704), chitosan microcapsules, and microsphere emulsions (U.S. Pat. No. 6,190,700).

In one embodiment, a composition for sustained bioavailability of a VEGF-R2 inhibitor comprises a gene therapy construct comprising a gene therapy vectors, for example a gene therapy vector described herein below.

Viral Gene Therapy Vectors. In one embodiment, viral vectors of the invention are disabled, e.g. replication-deficient. That is, they lack one or more functional genes required for their replication, which prevents their uncontrolled replication in vivo and avoids undesirable side effects of viral infection. In one embodiment, all of the viral genome is removed except for the minimum genomic elements required to package the viral genome incorporating the therapeutic gene into the viral coat or capsid. For example, it is desirable to delete all the viral genome except: (a) the Long Terminal Repeats (LTRs) or Invented Terminal Repeats (ITRs); and (b) a packaging signal. In the case of adenoviruses, deletions are typically made in the E1 region and optionally in one or more of the E2, E3, and/or E4 regions. Other viral vectors can be similarly deleted of genes required for replication. Deletion of sequences can be achieved by recombinant means, for example, involving digestion with appropriate restriction enzymes, followed by religation. Replication-competent self-limiting or self-destructing viral vectors can also be used.

Nucleic acid constructs of the invention can be incorporated into viral genomes by any suitable means known in the art. Typically, such incorporation is performed by ligating the construct into an appropriate restriction site in the genome of the virus. Viral genomes can then be packaged into viral coats or capsids using any suitable procedure. In particular, any suitable packaging cell line can be used to generate viral vectors of the invention. These packaging lines complement the replication-deficient viral genomes of the invention, as they include, for example by incorporation into their genomes, the genes which have been deleted from the replication-deficient genome. Thus, the use of packaging lines allows viral vectors of the invention to be generated in culture.

Suitable packaging lines for retroviruses include derivatives of PA317 cells, ψ-2 cells, CRE cells, CRIP cells, E-86-GP cells, and 293GP cells. In one embodiment, 293 cells are employed for use with adenoviruses and adeno-associated viruses.

Plasmid Gene Therapy Vectors. A soluble VEGF-R receptor can also be encoded by a plasmid. Advantages of a plasmid carrier include low toxicity and easy large-scale production. A polymer-coated plasmid can be delivered using electroporation as described by Fewell et al., 2001. Alternatively, a plasmid can be combined with an additional carrier, for example a cationic polyamine, a dendrimer, or a lipid, that facilitates delivery. See e.g., Baher et al., 1999; Maruyama-Tabata et al., 2000; and Tam et al., 2000.

Liposomes. A VEGF-R2 inhibitor of the present invention can also be delivered using a liposome. For example, a recombinantly produced soluble VEGF-R polypeptide can be encapsulated in liposomes. Liposomes can be prepared by any of a variety of techniques that are known in the art. See e.g., Dracopoli et al., 1994; Lasic & Martin, 1995; Janoff, 1999; Gregoriadis, 1993; Betageri et al., 1993; and U.S. Pat. Nos. 4,235,871; 4,551,482; 6,197,333; and 6,132,766. Temperature-sensitive liposomes can also be used, for example THERMOSOMES™ as disclosed in U.S. Pat. No. 6,200,598. Entrapment of a VEGF-R2 inhibitor within liposomes of the present invention can be carried out using any conventional method in the art. In preparing liposome compositions, stabilizers such as antioxidants and other additives can be used.

Other lipid carriers can also be used in accordance with the claimed invention, such as lipid microparticles, micelles, lipid suspensions, and lipid emulsions. See e.g., Labat-Moleur et al., 1996; and U.S. Pat. Nos. 5,011,634; 6,056,938; 6,217,886; 5,948,767; and 6,210,707.

III.B. Targeting Ligands

As desired, a composition of the invention can include one or more ligands having affinity for a specific cellular marker to thereby enhance delivery of a VEGF-R2 inhibitor to a tumor in vivo. Ligands include, but are not limited to antibodies, cell surface markers, peptides, and the like, which act to home the VEGF-R2 inibitor to a tumor, including the tumor vasculature.

The terms “targeting” and “homing”, as used herein to describe the in vivo activity of a ligand following administration to a subject, each refer to the preferential movement and/or accumulation of a ligand in a target tissue (e.g., a tumor) as compared with a control tissue.

The term “target tissue” as used herein refers to an intended site for accumulation of a ligand following administration to a subject. For example, the methods of the present invention employ a target tissue comprising a tumor.

The term “control tissue” as used herein refers to a site suspected to substantially lack binding and/or accumulation of an administered ligand. For example, in accordance with the methods of the present invention, a non-cancerous tissue is a control tissue.

The terms “selective targeting” of “selective homing” as used herein each refer to a preferential localization of a ligand that results in an amount of ligand in a target tissue that is in one embodiment about 2-fold greater than an amount of ligand in a control tissue, in another embodiment an amount that is about 5-fold or greater, and in still another embodiment an amount that is about 10-fold or greater. The terms “selective targeting” and “selective homing” also refer to binding or accumulation of a ligand in a target tissue concomitant with an absence of targeting to a control tissue, in one embodiment the absence of targeting to all control tissues.

The terms “targeting ligand” and “targeting molecule” as used herein each refer to a ligand that displays targeting activity. In one embodiment, a targeting ligand displays selective targeting. Representative targeting ligands include peptides and antibodies.

The term “peptide” encompasses any of a variety of forms of peptide derivatives, that include, but are not limited to amides, conjugates with proteins, cyclized peptides, polymerized peptides, conservatively substituted variants, analogs, fragments, peptoids, chemically modified peptides, and peptide mimetics. Representative peptide ligands that show tumor-binding activity include, for example, those described in U.S. Pat. Nos. 6,180,084 and 6,296,832.

The term “antibody” indicates an immunoglobulin protein, or functional portion thereof, including a polyclonal antibody, a monoclonal antibody, a chimeric antibody, a hybrid antibody, a single chain antibody (e.g., a single chain antibody represented in a phage library), a mutagenized antibody, a humanized antibody, and antibody fragments that comprise an antigen binding site (e.g., Fab and Fv antibody fragments). Representative antibody ligands that can be used in accordance with the methods of the present invention include antibodies that bind the tumor-specific antigens Her2/neu (v-erb-b2 avian erythroblastic leukemia viral oncogene homologue 2; Kirpotin et al., 1997; Becerril et al., 1999) and antibodies that bind to CEA (carcinoembryonic antigen; Ito et al., 1991). See also U.S. Pat. Nos. 5,111,867; 5,632,991; 5,849,877; 5,948,647; 6,054,561 and PCT International Publication No. WO 98/10795.

In an effort to identify ligands that are capable of targeting to multiple tumor types, targeting ligands have been developed that bind to target molecules present on tumor vasculature (Baillie et al., 1995; Pasqualini & Ruoslahti, 1996; Arap et al, 1998; Burg et al., 1999; Ellerby et al., 1999).

A targeting ligand can also comprise a ligand that specifically binds to a radiation induced target molecule. Ionizing radiation induces proteins in tumor vascular endothelium through transcriptional induction and/or posttranslational modification of cell adhesion molecules such as integrins (Hallahan et al., 1995; Hallahan et al., 1996; Hallahan et al., 1998; Hallahan & Virudachalam, 1999). For example, radiation induces activation of the integrin α_{2b}β₃, also called the fibrinogen receptor, on platelets. The induced molecules can serve as binding sites for targeting ligands. A representative peptide ligand that binds to irradiated tumors comprises Biapcitide (ACUTECT® available from Diatide, Inc., Londonberry, N.H., United States of America), which specifically binds to GP-IIb/IIIa receptors on activated platelets (Hawiger et al., 1989; Hawiger & Timmons, 1992; Hallahan et al., 2001).

Antibodies, peptides, or other ligands can be coupled to drugs (e.g., a VEGF-R2 inhibitor) or drug carriers using methods known in the art, including but not limited to carbodiimide conjugation, esterification, sodium periodate oxidation followed by reductive alkylation, and glutaraldehyde crosslinking. See e.g., Bauminger & Wilchek, 1980; Goldman et al., 1997; Kirpotin et al., 1997; Dracopoli et al., 1994; Neri et al., 1997; Park et al., 1997; and Pasqualini et al., 1997; U.S. Pat. No. 6,071,890; and European Patent No. 0 439 095. Alternatively, pseudotyping of a retrovirus can be used to target a virus towards a particular cell (Marin et al., 1997).

III.C. Formulation

In one embodiment, a composition of the present invention comprises a VEGF-R2 inhibitor and a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, for example in the range of in one embodiment about 0.1 to 10 mg/ml, in another embodiment about 2.0 mg/ml; and/or mannitol or another sugar, for example in the range of in one embodiment 10 to 100 mg/ml, in another embodiment about 30 mg/ml; phosphate-buffered saline (PBS), and any other formulation agents conventional in the art.

The therapeutic regimens and pharmaceutical compositions of the invention can be used with additional adjuvants or biological response modifiers including, but not limited to, the cytokines interferon alpha (IFN-α), interferon gamma (IFN-γ), interleukin 2 (IL-2), interleukin 4 (IL-4), interleukin 6 (IL-6), tumor necrosis factor (TNF), or other cytokine affecting immune cells.

IV. Therapeutic Methods

The disclosed VEGF-R2 inhibitors are useful as radiosensitizing agents. Thus, the present invention provides a method for treating a tumor comprising: (a) administering a VEGF-R2 inhibitor to a subject bearing a tumor; whereby the radiosensitivity of the tumor is increased; and (b) treating the tumor with ionizing radiation, whereby tumor growth is delayed. Also provided is a method for inhibiting tumor blood vessel growth and/or delaying tumor growth via administration of a VEGF-R2 inhibitor. In one embodiment, the performance of the method also promotes tumor regression.

While applicants do not intend to be bound by any particular theory of operation, a VEGF-R2 inhibitor may effectively delay tumor growth by blocking reprofusion of an irradiated tumor. Specifically, a VEGF-R2 inhibitor can block processes that require VEGF, including endothelial cell infiltration and budding of tumor blood vessels.

IV.A. Administration of a VEGF-R2 Inhibitor

Suitable methods for administration of a composition of the present invention include but are not limited to intravascular, subcutaneous, or intratumoral administration. For delivery of compositions to pulmonary pathways, compositions can be administered as an aerosol or coarse spray. A delivery method is selected based on considerations including, but not limited to the type of VEGF-R2 inhibitor, the type of carrier or vector, toxicity of the VEGF-R2 inhibitor, therapeutic efficacy of the VEGF-R2 inhibitor, and the condition of the tumor to be treated. In one embodiment of the invention, intravascular administration is employed.

In one embodiment, a therapeutically effective amount of a composition of the invention is administered to a subject. A “therapeutically effective amount” is an amount of a composition comprising a VEGF-R2 inhibitor sufficient to produce a measurable anti-tumor response (e.g., an anti-angiogenic response, a cytotoxic response, and/or tumor regression). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. Determination and adjustment of a therapeutically effective dose, as well as evaluation of when and how to make such adjustments, are known to those of ordinary skill in the art of medicine.

In one embodiment of the invention, a minimally therapeutic dose of a VEGF-R2 inhibitor is administered. The term “minimally therapeutic dose” refers to the smallest dose, or smallest range of doses, determined to be a therapeutically effective amount as defined herein above.

For local administration of viral vectors, previous clinical studies have demonstrated that up to 10¹³ pfu of virus can be injected with minimal toxicity. In human patients, 1×10⁹−1×10¹³ pfu are routinely used. See Habib et al., 1999. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor and/or anti-angiogenic activity. Representative criteria and methods for assessing anti-tumor and/or anti-angiogenic activity are described herein below.

For small molecule VEGF-R2 inhibitors and soluble formulations of VEGF-R2 inhibitors, conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses are also given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, the dose is multiplied by the appropriate km factor. In adult humans, 100 mg/kg is equivalent to 100 mg/kg×37 kg/m²=3700 mg/m².

Additional formulation and dose techniques have been described in the art, see for example, those described in U.S. Pat. Nos. 5,326,902 and 5,234,933, and PCT International Publication No. WO 93/25521.

IV.B. Radiation Treatment

For treatment of a radiosensitized tumor, the tumor is irradiated concurrent with, or subsequent to, administration of a composition comprising a VEGF-R2 receptor. In one embodiment of the present invention, the tumor is irradiated daily for 2 weeks to 7 weeks (for a total of 10 treatments to 35 treatments). Alternatively, tumors can be irradiated with brachytherapy utilizing high dose rate or low dose rate brachytherapy internal emitters.

The duration for administration of a VEGF-R2 inhibitor comprises in one embodiment a period of several months coincident with radiotherapy, but in another embodiment can extend to a period of 1 year to 3 years as needed to effect tumor control. A VEGF-R2 inhibitor comprising a small compound (e.g., SU5416) can be administered about one hour before each fraction of radiation. A gene therapy vector encoding a VEGF-R2 inhibitor (e.g., Ad.ExFlk) can be administered prior to an initial radiation treatment and then at desired intervals during the course of radiation treatment (e.g., weekly, monthly, or as required). An initial administration of a VEGF-R2 inhibitor comprising a gene therapy vector or a sustained release drug carrier can comprise administering the VEGF-R2 inhibitor to a tumor during placement of a brachytherapy afterloading device.

Subtherapeutic or therapeutic doses of radiation can be used for treatment of a radiosensitized tumor as disclosed herein. In one embodiment, a subtherapeutic or minimally therapeutic dose (when administered alone) of ionizing radiation is used. For example, the dose of radiation can comprise in one embodiment at least about 2 Gy ionizing radiation, in another embodiment about 2 Gy to about 6 Gy ionizing radiation, and in still another embodiment about 2 Gy to about 3 Gy ionizing radiation. When radiosurgery is used, representative doses of radiation include about 10 Gy to about 20 Gy administered as a single dose during radiosurgery or about 7 Gy administered daily for 3 days (about 21 Gy total). When high dose rate brachytherapy is used, a representative radiation dose comprises about 7 Gy daily for 3 days (about 21 Gy total). For low dose rate brachytherapy, radiation doses typically comprise about 12 Gy administered twice over the course of 1 month. ¹²⁵I seeds can be implanted into a tumor can be used to deliver very high doses of about 110 Gy to about 140 Gy in a single administration.

Radiation can be localized to a tumor using conformal irradiation, brachytherapy, stereotactic irradiation, or intensity modulated radiation therapy (IMRT). The threshold dose for treatment can thereby be exceeded in the target tissue but avoided in surrounding normal tissues. For treatment of a subject having two or more tumors, local irradiation enables differential drug administration and/or radiotherapy at each of the two or more tumors. Alternatively, whole body irradiation can be used, as permitted by the low doses of radiation required following radiosensitization of the tumor.

Radiation can also comprise administration of internal emitters, for example ¹³¹I for treatment of thyroid cancer, NETASTRON™ and QUADRAGEN®) pharmaceutical compositions (Cytogen Corp., Princeton, N.J., United States of America) for treatment of bone metastases, ³²P for treatment of ovarian cancer. Other internal emitters include ¹²⁵I, iridium, and cesium. Internal emitters can be encapsulated for administration or can be loaded into a brachytherapy device.

Radiotherapy methods suitable for use in the practice of this invention can be found in Leibel & Phillips, 1998, among other sources.

### EXAMPLES

The following Examples have been included to illustrate modes of the invention. Certain aspects of the following Examples are described in terms of techniques and procedures found or contemplated by the present inventor to work well in the practice of the invention. These Examples illustrate standard laboratory practices of the inventor. In light of the present disclosure and the general level of skill in the art, those of skill will appreciate that the following Examples are intended to be exemplary only and that numerous changes, modifications, and alterations can be employed without departing from the scope of the invention.

### Example 1

Tumor Models

The B16F0 cell line was obtained from American Type Culture Collection (ATCC; Manassas, Va., United States of America). B16F0 cells were maintained in HYCLONE® minimal essential culture medium (Hyclone Laboratories, Logan, Utah, United States of America), supplemented with 10% fetal calf serum and 0.5% penicillin-streptomycin. GL261 cells were maintained in a 1:1 mixture of Dulbecco's Modified Eagle Medium (DMEM) and F-12 nutrient mixture 1:1, supplemented with 7% fetal calf serum, 0.5% penicillin/streptomycin, and 1% sodium pyruvate. HYCLONEN® culture medium was purchased from VWR, West Chester, Pa., United States of America. DMEM, F-12 nutrient mixture, fetal calf serum, and penicillin/streptomycin were purchased from GIBCO™ Invitrogen Corporation of Carlsbad, Calif., United States of America.

The GL261 cell line was obtained from Yancie Gillespie (University of Alabama, Birmingham, Ala., United States of America) and is described by Hallahan et al., 1998; Staba et al., 1998; and Baumann et a/., 1992. GL261 cells form tumors in C57BL6J mice following subcutaneous injection into hind limb (Hallahan et al., 1998) and into a dorsal skin fold window chamber. Cells were trypsinized and counted using a hemocytometer. Cells were washed in complete medium and 10⁶ cells were injected subcutaneously into the hind limb or into the dorsal skin fold window.

The GL216 glioblastoma model shows high levels of VEGF expression and radiation resistance in vivo. See Gorski et al., 1999.

### Example 2

Tumor Vascular Window Model

Penicillin/streptomycin solution (200 μl; available from GIBCO™ Invitrogen Corporation, Carlsbad, Calif., United States of America) was injected into the hind limb of the mouse prior to preparation of the window chamber. The dorsal ventral window chamber was prepared using a 3-gram plastic frame, which was applied to the skin of a test animal and remained attached for the duration of the study. Briefly, the chamber was screwed together, and the epidermis was cut and left open with a plastic covering.

To prepare a test animal for application of the plastic frame, the dorsal midline was found along the animal's back, and a clip was placed to hold the skin in position. A template, equivalent to the outer diameter of the chamber was traced, producing the outline of the incision. A circular cut was made tracing the perimeter of the outline (a circular area about 7 mm in diameter) followed by a crisscross cut through the center of the circular area, thus producing four skin flaps. The epidermis of the four flaps was removed using a scalpel with an effort to follow the hypodermis superior to the fascia. The area was then trimmed and manicured with a pair of fine forceps and iris scissors. During surgery, the area was kept moist by applying moist drops of phosphate buffered saline with 1% penicillin/streptomycin solution.

The template was removed and the top piece of the chamber was fixed with screws. The bottom portion of the chamber was put in place and the top portion of the chamber was carefully positioned on the cut side, so that the window and the circular incision were fitted. Antibiotic ointment was applied to keep the area clear of infection. The three screws that hold the chamber together, were then put into the chamber holes and tightened so that the skin was not pinched, thus avoiding diminished circulation.

GL261 and B16F0 cells were implanted into the dorsal skin fold window chamber. Blood vessels developed over 1 week. Tumor vasculature was observed in response to various treatments as described below. To document changes in tumor blood vessels, the window frame was marked with coordinates, which were used to photograph the same microscopic field each day beginning at day 0 through day 7. Vascular windows were photographed using a 4× objective to obtain a 40× total magnification. Color slides were scanned into PHOTOSHOP® software (Adobe Systems, Inc., San Jose, Calif., United States of America). Scanned slides were analyzed using OPTIMAS® image analysis software (Bioscan, Inc., Edmonds, Wash., United States of America; available from Media Cybernetics, Silver Spring, Md., United States of America) or IMAGE-PRO® software (Media Cybernetics, Silver Spring, Md., United States of America) as described below.

Vascular center lines were positioned by OPTIMAS® software or IMAGE-PRO® software and verified by an observer blinded to the treatment groups. Tumor blood vessels were quantified using OPTIMAS® software, which quantified the vascular length density of blood vessel within the microscopic field. The mean vascular length density and the standard error of the mean were calculated for each treatment group. Variance was analyzed using the Kruskal-Wallis method. See Kruskal & Wallis, 1952 and Hallahan et al., 1995.

The vascular window model allowed direct measurement of the vascular response to ionizing radiation. Window models were prepared using the GL261 and B16F0 tumor cell lines. Animals were treated with 2 Gy ionizing radiation, 3 Gy ionizing radiation, or 6 Gy ionizing radiation. Photographs of blood vessels taken prior to and each day subsequent to radiation treatment were used for analysis.

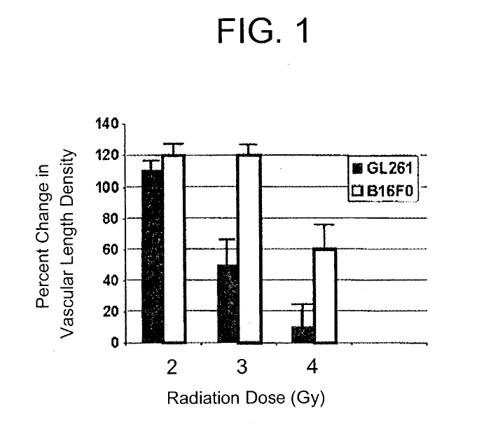

A dose- and time-dependent reduction of tumor vasculature was observed in response to radiation treatment. B16F0 tumor blood vessels showed a slight increase in vascular length density following treatment with 2 Gy ionizing radiation. Treatment with 3 Gy reduced tumor vascular length density within 48 hours (probability p<0.05), and 6 Gy eliminated vasculature within 24 hours. Blood vessels within the GL261 glioma showed increased vascular length density in response to 3 Gy ionizing radiation, and only a minimal reduction in vascular length density in response to 6 Gy ionizing radiation (p=0.04, measured at 72 hours following radiation treatment). Minimal variation in radiation response was observed among members within a treatment group. See FIG. 1.

### Example 3

Power Doppler Sonography

Power Doppler sonography was used to study the response of tumor blood vessels to ionizing radiation. This method utilizes amplitude to measure flow in microvasculature. Tumors were imaged with a 10-5 MHz ENTOS® linear probe (Advanced Technology Laboratories, Inc., Bothell, Wash., United States of America) attached to an HDI® 5000 diagnostic ultrasound system (Advanced Technology Laboratories, Inc., Bothell, Wash., United States of America).

Power Doppler sonography images were obtained with the power gain set to 82%. Care was taken to minimize motion artifact. A 20-frame cineloop sweep of the entire tumor was obtained with the probe perpendicular to the long axis of the lower extremity along the entire length of the tumor.

The images were analyzed using HDI® software (Advanced Technology Laboratories, Inc., Bothell, Wash., United States of America). This software allows direct evaluation of Power Doppler cineloop raw data. Color area was recorded for the entire tumor. Five mice were included in each treatment group. Values for color area were averaged for each tumor set and treated groups were compared to controls using the unpaired student t-test.

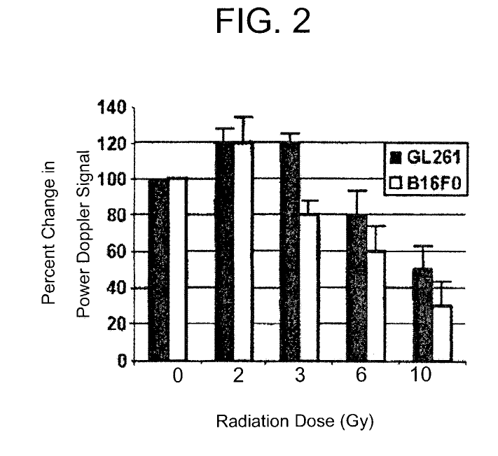

B16F0 melanoma and GL261 glioma tumors were implanted in the hind limb of C57BL6J mice. Tumors were grown to a diameter of about 0.7 cm to about 1.1 cm and then irradiated with 2 Gy ionizing radiation, 3 Gy ionizing radiation, 6 Gy ionizing radiation, or 10 Gy ionizing radiation. Power Doppler analysis of tumor blood flow was measured on day 0, day 3, and day 7 after radiation treatment. See FIG. 2.

Blood flow increased following low dose irradiation (3 Gy), and blood flow decreased following high dose irradiation. The flow in each pixel (Power weighted pixel density) was summed to compare dose-dependent and tumor-dependent changes in tumor blood flow over time. Blood flow throughout the entire tumor was measured after treatment with 6 Gy ionizing radiation. Blood flow decreased by day 3 (p=0.045). At day 7, blood flow in B16F0 tumors was recovered, and blood flow in GL261 tumors was greater than prior to treatment.

### Example 4

VEGF-R2 Inhibitors

Flk-1 is a receptor tyrosine kinase that is specifically inhibited by the dominant negative function of a soluble and truncated form of the receptor, which is called soluble Flk-1 receptor. A soluble Flk-1 receptor was constructed by fusing a sequence encoding the extracellular domain of murine Flk-1 to a sequence encoding a 6-histidine tag at the carboxyl terminus. An adenoviral construct encoding the fusion protein is referred to as “Ad.ExFlk.6His,” and the encoded fusion protein is referred to as “ExFlk.6His.”

ExFlk.6His blocks activation of Flk-1 and formed heterodimers with endogenous cell surface Flk-1 in the presence of VEGF. ExFlk.6His also inhibits VEGF-induced DNA synthesis and migration in HUVEC. See Lin et al., 1998.

SU5416 is a potent and selective inhibitor of Flk-1/KDR that blocks tyrosine kinase catalysis and inhibits tumor vascularization and growth (Fong et al., 1999; Shaheen et al., 1999; Vajkoczy et al., 1999). The chemical formula is 3-[2,4-dimethyl pyrrol-5-yl) methyl idenyl]-2-indolinone, and the compound has an inhibition constant (Kᵢ) of 0.16 μM for Flk-1. The plasma half-life of SU5416 is relatively short (1-2 hours), but the biological effectiveness of this agent is durable and capable to inhibit VEGF-R2 for greater than 3 days. Thus, for combined Flk-1 inhibition and radiation treatments (Examples 5 and 7), SU5416 was administered at a frequency less than the standard radiation course.

### Example 5

Flk-1 Antagonists Eliminate Radioresistance

To determine whether the Flk-1 receptor inhibition alters the radiation response in tumor blood vessels, window model analysis and Power Doppler blood flow analysis were performed in animals treated with VEGF-R2 inhibitors followed by radiotherapy.

To determine whether the Flk-1 receptor inhibition alters the radiation response in tumor blood vessels, GL261 and B16F0 tumors were established in C57BL6 mice, and vascular length density and blood flow were studied following administration of a Flk-1 inhibitor in combination with radiotherapy. Ad.ExFlk.6His and control construct Ad.LacZ were administered to tumor bearing mice at a minimally effective dose of 2×10⁸ pfu by tail vein injection, and a subtherapeutic dose of radiation (2 Gy) was administered 24 hours later. To determine whether Flk-1 kinase inhibitors enhance radiation cytotoxicity, a minimal effective dose of SU5416 (0.75 mg) was administered in combination with a subtherapeutic dose of radiation (2 Gy).

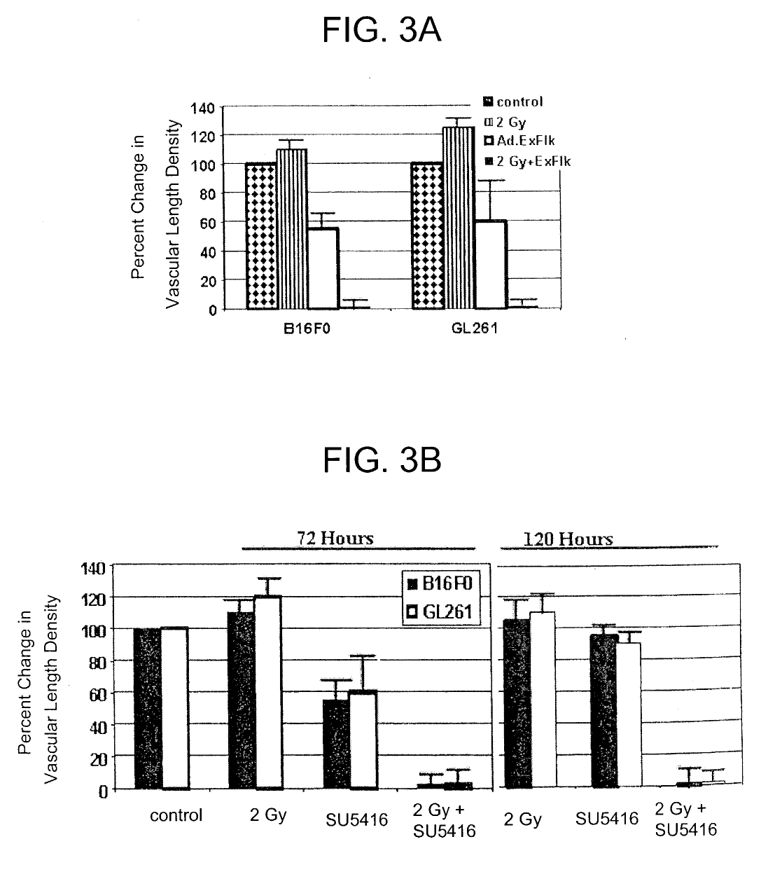

Following treatment of animals by administration of Ad.ExFlk in combination with radiotherapy, vascular regression was studied as described in Example 2. No vascular regression was achieved following treatment with 2 Gy and control construct Ad.lacZ. GL261 and B16F0 tumors treated with 2 Gy alone showed no vascular response to radiation. Administration of Ad.ExFlk.6His at 2×10⁸ pfu, in the absence of radiation treatment, achieved minimal regression of tumor blood vessels. When Ad.ExFlk.6His was administered 16 hours prior to exposure to 2 Gy ionizing radiation, tumor blood vessels regressed at a rate typically observed following treatment with 6 Gy. Enhanced anti-angiogenic effects following combined Ad.ExFlk.6His and radiation treatments were observed in both tumor models. See FIG. 3A.

Similarly, administration of 0.75 mg SU5416, in the absence of ionizing radiation, resulted in a minimal regression of the tumor blood vessels and rapid restoration of vasculature. SU5416 administered 1 hour prior to treatment with 2 Gy ionizing radiation completely destroyed tumor blood vessels within 24 hours of treatment (p=0.02, as compared to radiation alone). Enhanced anti-angiogenic effects following combined SU5416 and radiation treatments were observed in both tumor models. See FIG. 3B.

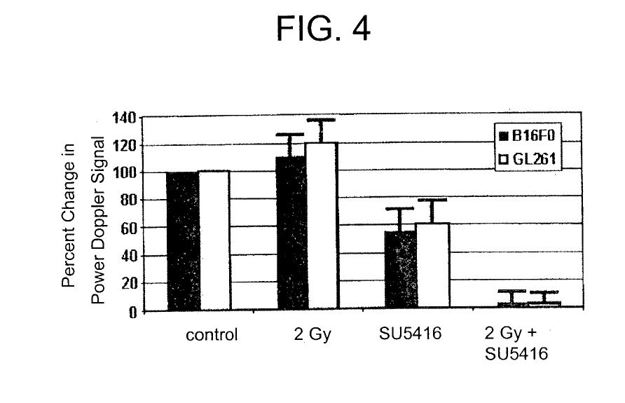

To determine whether inhibition of Flk-1-mediated signal transduction also enhances the cytotoxic effects of radiotherapy in established tumors, Power Doppler sonography was used to quantify blood flow in 1 cm diameter tumors treated with Flk-1 inhibitors and ionizing radiation. Tumor bearing mice were treated with a minimally effective dose of SU5416 (0.75 mg) administered by intraperitoneal injection at 1 hour prior to administration of 2 Gy ionizing radiation. SU5416 was injected every other fraction for a total of 3 doses. Radiation was delivered in fraction doses of 3 Gy per day for 7 days. Blood flow was quantified by Power Doppler sonography on days 4 and 7 according to methods described in Example 3. Treatment with radiation alone and SU5416 alone achieved minimal reduction in GL261 and B16F0 tumor blood flow. Tumors treated with SU5416 one hour prior to irradiation showed a significant reduction in tumor blood flow as compared to either treatment alone (p=0.004), consistent with observations of enhanced effect of combined treatment when using the window model. See FIG. 4.

### Example 6

Flk-1 Inhibition Abrogates Endothelial Cell Survival

VEGF prolongs the survival of human endothelial cells, which is associated with increased expression of the anti-apoptotic protein Bcl-2 (Gerber et al., 1998; Nor et al., 1999). Enhanced endothelial cell survival was associated with a dose-dependent increase in Bcl-2 expression and decrease in expression of the processed forms of caspase-3 (Nor et al., 1999). Over-expression of Bcl-2 in HUVEC prevented apoptotic cell death in the absence of VEGF (Gerber et al., 1998). Although it is not applicant's intention to be bound by any particular theory of operation, Flk-1 inhibitors appear to modify a subsequent response to radiotherapy at least in part, although not necessarily exclusively, via regulation of apoptosis.

To determine whether Flk-1 inhibitors enhance radiation-induced intrinsic cell death mechanisms, radiation-induced apoptosis in HUVEC human primary culture endothelial cells, 3B11 murine endothelial cells, and human microdermal endothelial cells (HMEC) was studied following Flk-1 inhibition. HMEC cells and 3B11 cells were treated with 50 μm SU5416 or with 10⁸ pfu Ad.ExFlk or control vector Ad.LacZ. Cells were then treated with 6 Gy of ionizing radiation. Cultures were stained to reveal apoptotic cells 24 hours later. Endothelial cell death was quantified by counting the number of apoptotic bodies in 100 cells.

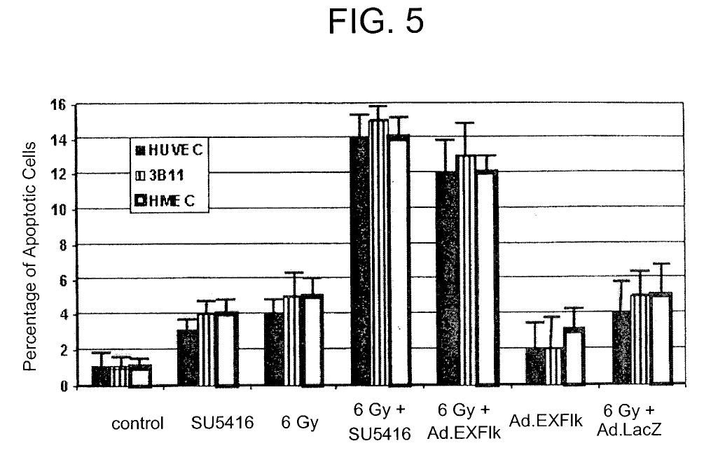

Endothelial cells showed minimal radiation-induced apoptosis in complete medium. When soluble Flk-1 receptor (Ad.ExFlk) or SU5416 were included in the medium, endothelial cells showed radiation-induced apoptosis. The percentage of HUVEC undergoing apoptosis following exposure to ionizing radiation was 2%. No apoptotic cells were observed in the population of control cells not exposed to ionizing radiation. Cells exposed to SU5416 or to Ad.ExFlk, but not exposed to ionizing radiation, induced apoptosis in 4% of cells and 5% of cells, respectively. When Flk-1 inhibitors were added to the culture medium prior to exposure of cells to ionizing radiation, increased apoptosis was observed. HUVEC cultures showed 14% apoptotic cells in response to SU5416 treatment following radiation treatment (p=0.005 compared to radiation alone). Similarly, HUVEC cultures treated with Ad.ExFlk following by ionizing radiation showed 12% apoptotic cells (p=0.01 compared to radiation alone). 3B11 and HMEC cells treated with Flk-1 inhibitors and radiation showed a similar enhancement of radiation-induced apoptosis. See FIG. 5.

### Example 7

Tumor Volume Assessment

GL261 tumors were generated by subcutaneous injection of 10⁶ viable GL261 cells suspended in 0.2 ml of a 0.6% agarose solution into the right thigh of C57BL6 mice. Mice were stratified into four groups so that the mean tumor volume of each group was comparable. An equal number of large and intermediate size tumors were present in each group. The mean tumor volumes in each group were as follows: radiation, 0.52 cm³; SU5416, 0.56 cm³; SU5416 followed by radiation, 0.59 cm³; untreated control, 0.49 cm³.

GL261 tumor bearing mice were treated with 3 Gy ionizing radiation on each of 3 days per week for two weeks. SU5416 was administered at 0.75 mg by intraperitoneal injection given twice per week for a total of 4 treatments on days 0, 4, 7, and 11. Radiation was administered as 3 Gy fractions on 8 occasions on days 0, 1, 3, 4, 7, 8, 10, and 11. Control mice received identical doses and schedule of either SU5416 alone or radiation alone.

Twice each week, tumor volumes were measured using skin calipers as previously described by Advani et al., 1998; Hallahan et al., 1995; and Staba et al., 1998. Tumor volumes were calculated using a formula:

a×b×c/2,

where a=maximal width dimension, b=maximal length dimension, and c=maximal depth dimension, which was derived from the formula for an ellipsoid (πd³/6). Data were calculated as the percent of original (day 0) tumor volume and graphed as fractional tumor volume ±1 standard deviation for each treatment group. Variance was analyzed by the Kruskal-Wallis method (Kruskal & Wallis, 1952).

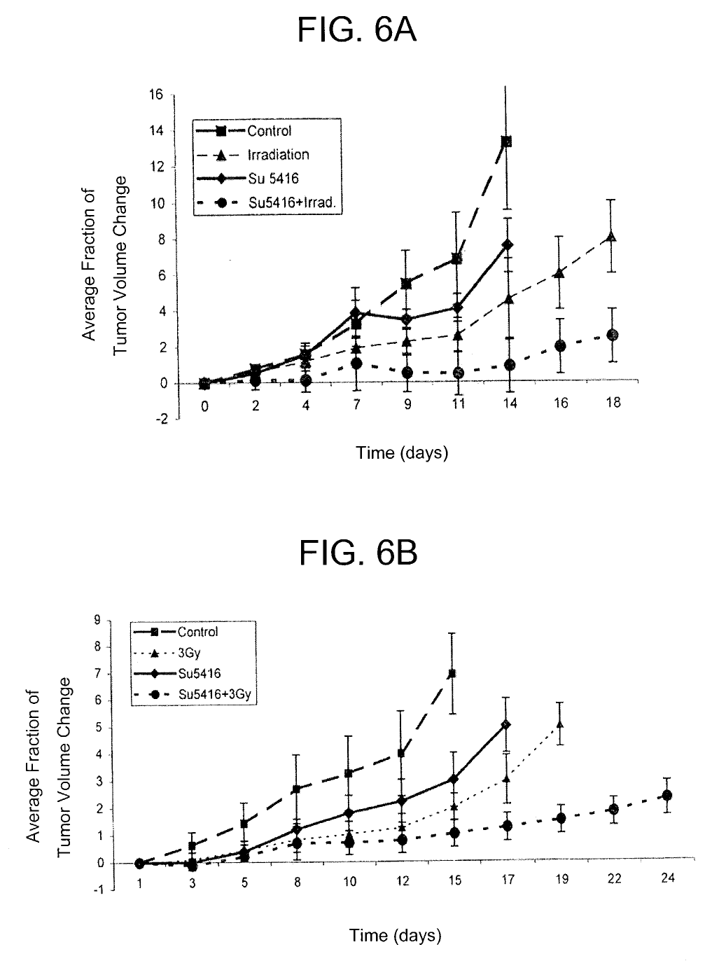

Growth delay was determined as the number of days required for a tumor to reach a predetermined volume, for example three-fold the original volume, or a volume of 1 cm³. GL261 tumors treated with radiation alone showed minimal growth delay. No growth delay was observed in tumors treated with SU5416 alone. However, the combination of SU5416 followed by radiation significantly reduced tumor volumes when compared to radiation alone (p=0.01) or SU5416 alone (p<0.009). See FIG. 6A.

In a second experiment, mice were randomly assigned to each of the four treatment groups for a total of 10 mice in each group. The mean tumor volume on day 1 for mice in each treatment group was as follows: radiation, 0.90 cm³; SU5416, 0.99 cm³; SU5416 followed by radiation, 0.90 cm³; untreated control, 0.91 cm³. Flk-1 inhibition and radiation treatments were initiated fourteen days after the tumor cells had been injected and were performed as described herein above. Tumors treated with SU5416 and radiation showed significantly reduced tumor volumes when compared to radiation alone (p=0.01) and to SU5416 alone (p=0.006). See FIG. 6B.

### Example 8

Restoration of Tumor Blood Flow After Radiotherapy

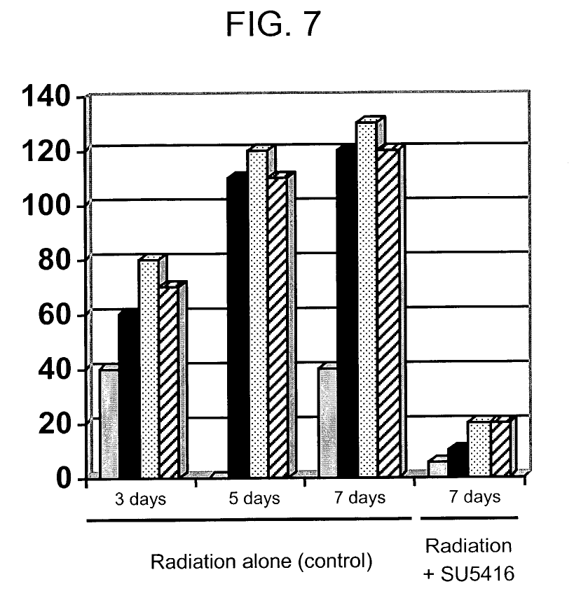

Tumor-bearing mice were generated as described in Example 1. Mice were treated with SU5416 in combination with radiation treatment as described in Example 5. Control animals were treated with radiation alone. Doppler analysis of tumor blood flow was measured daily for 7 days using the methods described in Example 3. In control animals receiving only radiation treatment, restoration of tumor blood flow was observed by day 7. In animals receiving SU5416 following radiation treatment, tumor blood flow was repressed (FIG. 7).

While applicants do not intend to be bound to any particular theory of operation, the mechanisms of tumor blood flow restoration appears to involve infiltration of a tumor by endothelial progenitor cells (EPCs). EPCs were isolated from peripheral circulation, fixed with paraformaldehyde, and incubated with antibodies that specifically bind to Flk or to CD31. A fluorescently-labeled secondary antibody was used to detect Flk and CD31 (PECAM; platelet-endothelial cell adhesion molecule 1) proteins on the surface of EPCs.

Mice bearing GI261 gliomas, generated as described in Example 1, received radiation NO: NO: treatment of the tumor. EPCs were isolated from peripheral circulation and fluorescently labeled with Dil (1,1′-dioctadecyl-3,3,3′,3′-tetramethylindocarbocyanine perchlorate; available from Molecular Probes, Inc., Eugene, Oreg., United States of America). Labeled EPCs were then administered via tail vein injection to tumor-bearing mice immediately following tumor irradiation. Tumor sections were obtained on days 1, 4, and 7 following irradiation. In control mice, EPCs were detected in the perivascular space at day 4, and then proliferated and migrated into to the tumor by day 7. Mice treated with SU5416 and radiation, extravasation of EPCs into the tumor was prevented.

References

The references listed below as well as all references cited in the specification are incorporated herein by reference to the extent that they supplement, explain, provide a background for or teach methodology, techniques and/or compositions employed herein.

Advani S J, Sibley G S, Song P Y, Hallahan D E, Kataoka Y, Roizman B & Weichselbaum R R (1998) Enhancement of Replication of Genetically Engineered Herpes Simplex Viruses by Ionizing Radiation: A New Paradigm for Destruction of Therapeutically Intractable Tumors. Gene Ther 5:160-165.

Allam A, Taghian A, Gioioso D, Duffy M & Suit H D (1993) Intratumoral Heterogeneity of Malignant Gliomas Measured in Vitro. NO: Int J Radiat Oncol Biol Phys 27:303-308.

Altschul S F, Gish W, Miller W, Myers E W & Lipman D J (1990) Basic Local Alignment Search Tool. J Mol Biol 215:403-410.

Arap W, Pasqualini R & Ruoslahti E (1998) Cancer Treatment by Targeted Drug Delivery to Tumor Vasculature in a Mouse Model. Science 279:377-380.

Ausubel F, ed (1995) Short Protocols in Molecular Biology, 3rd ed. Wiley, New York.

Baher A G, Andres M L, Folz-Holbeck J, Cao J D & Gridley D S (1999) A Model Using Radiation and Plasmid-Mediated Tumor Necrosis Factor-Alpha Gene Therapy for Treatment of Glioblastomas. Anticancer Res 19:2917-2924.

Baillie C T, Winslet M C & Bradley N J (1995) Tumour Vasculature—a Potential Therapeutic Target. Br J Cancer 72:257-267.

Barton G J (1998) Protein Sequence Alignment Techniques. Acta Crystallogr D Biol Crystallogr 54:1139-1146.

Batzer M A, Carlton J E & Deininger P L (1991) Enhanced Evolutionary PCR Using Oligonucleotides with Inosine at the 3′- Terminus. Nucleic Acids Res 19:5081.

Baumann M, DuBois W, Pu A, Freeman J & Suit H D (1992) Response of Xenografts of Human Malignant Gliomas and Squamous Cell Carcinomas to Fractionated Irradiation. Int J Radiat Oncol Biol Phys 23:803-809.

Bauminger S & Wilchek M (1980) The Use of Carbodiimides in the Preparation of Immunizing Conjugates. Methods Enzymol 70:151-159.

Becerril B, Poul M A & Marks J D (1999) Toward Selection of Internalizing Antibodies from Phage Libraries. Biochem Biophys Res Commun 255:386-393.

Betageri G V, Jenkins S A & Parsons D L (1993) Liposome Drug Delivery Systems. Technomic Pub., Lancaster, Pa., United States of America.

Burg M A, Pasqualini R, Arap W, Ruoslahti E & Stallcup W B (1999) NG2 Proteoglycan-Binding Peptides Target Tumor Neovasculature. Cancer Res 59:2869-2874.

Dracopoli N C, Haines J L, Korf B R, Morton C C, Seidman C E, Seidman J G & Smith D R, eds (1994) Current Protocols in Human Genetics on CD-ROM. John Wiley & Sons, New York.

Ellerby H M, Arap W, Ellerby L M, Kain R, Andrusiak R, Rio GD, Krajewski S, Lombardo C R, Rao R, Ruoslahti E, Bredesen D E & Pasqualini R (1999) Anti-Cancer Activity of Targeted Pro-Apoptotic Peptides. Nat Med 5:1032-1038.

EP 0 439 095

Fewell J G, MacLaughlin F, Mehta V, Gondo M, Nicol F, Wilson E & Smith L C (2001) Gene Therapy for the Treatment of Hemophilia B Using PINC-Formulated Plasmid Delivered to Muscle with Electroporation. Mol Ther 3:574-583.

Fong T A, Shawver L K, Sun L, Tang C, App H, Powell T J, Kim Y H, Schreck R, Wang X, Risau W, Ullrich A, Hirth K P & McMahon G (1999) Su5416 Is a Potent and Selective Inhibitor of the Vascular Endothelial Growth Factor Receptor (Flk-1/KDR) That Inhibits Tyrosine Kinase Catalysis, Tumor Vascularization, and Growth of Multiple Tumor Types. Cancer Res 59:99-106.

Freireich E J, Gehan E A, Rall D P, Schmidt L H & Skipper H E (1966) Quantitative Comparison of Toxicity of Anticancer Agents in Mouse, Rat, Hamster, Dog, Monkey, and Man. Cancer Chemother Rep 50:219-244.

Gerber H P, Dixit V & Ferrara N (1998) Vascular Endothelial Growth Factor Induces Expression of the Antiapoptotic Proteins Bcl-2 and A1 in Vascular Endothelial Cells. J Biol Chem 273:13313-13316.

Glover D M & Hames B D (1995) DNA Cloning: A Practical Approach, 2nd ed. IRL Press at Oxford University Press, Oxford/N.Y.

Goldman C K, Rogers B E, Douglas J T, Sosnowski B A, Ying W, Siegal G P, Baird A, Campain J A & Curiel D T (1997) Targeted Gene Delivery to Kaposi's Sarcoma Cells Via the Fibroblast Growth Factor Receptor. Cancer Res 57:1447-1451.

Goldman C K, Kendall R L, Cabrera G, Soroceanu L, Heike Y, Gillespie G Y, Siegal G P, Mao X, Bett A J, Huckle W R, Thomas K A & Curiel D T (1998) Paracrine Expression of a Native Soluble Vascular Endothelial Growth Factor Receptor Inhibits Tumor Growth, Metastasis, and Mortality Rate. Proc Natl Acad Sci U S A 95:8795-8800.

Gorski D H, Beckett M A, Jaskowiak N T, Calvin D P, Mauceri H J, Salloum R M, Seetharam S, Koons A, Hari D M, Kufe D W & Weichselbaum R R (1999) Blockage of the Vascular Endothelial Growth Factor Stress Response Increases the Antitumor Effects of Ionizing Radiation. Cancer Res 59:3374-3378.

Gregoriadis G (1993) Liposome Technology, 2nd ed. CRC Press, Boca Raton, Fla., United States of America.

Griscelli F, Li H, Cheong C, Opolon P, Bennaceur-Griscelli A, Vassal G, Soria J, Soria C, Lu H, Perricaudet M & Yeh P (2000) Combined Effects of Radiotherapy and Angiostatin Gene Therapy in Glioma Tumor Model. Proc Natl Acad Sci U S A 97:6698-6703.

Habib N A, Hodgson H J, Lemoine N & Pignatelli M (1999) A Phase NO: I/II Study of Hepatic Artery Infusion with wtp53-CMV-Ad in Metastatic Malignant Liver Tumours. Hum Gene Ther 10:2019-2034.

Hallahan D, Kuchibhotla J & Wyble C (1996) Cell Adhesion Molecules Mediate Radiation-Induced Leukocyte Adhesion to the Vascular Endothelium. Cancer Res 56:5150-5155.

Hallahan D, Clark E T, Kuchibhotla J, Gewertz B L & Collins T (1995a) E-Selectin Gene Induction by Ionizing Radiation Is Independent of Cytokine Induction. Biochem Biophys Res Commun 217:784-795.

Hallahan D E & Virudachalam S (1999) Accumulation of P-Selectin in the Lumen of Irradiated Blood Vessels. Radiat Res 152:6-13.

Hallahan D E, Staba-Hogan M J, Virudachalam S & Kolchinsky A (1998) X-Ray-Induced P-Selectin Localization to the Lumen of Tumor Blood Vessels. Cancer Res 58:5216-5220.

Hallahan D E, Geng L, Cmelak A J, Chakravarthy A B, Martin W, Scarfone C & Gonzalez A (2001) Targeting Drug Delivery to Radiation-Induced Neoantigens in Tumor Microvasculature. J Control Release 74:183-191.

Hallahan D E, Mauceri H J, Seung L P, Dunphy E J, Wayne J D, Hanna N N, Toledano A, Hellman S, Kufe D W & Weichselbaum R R (1995b) Spatial and Temporal Control of Gene Therapy Using Ionizing Radiation. Nat Med 1:786-791.

Harlow E & Lane D (1988) Antibodies: A Laboratory Manual. Cold Spring Harbor Laboratory Press, Cold Spring Harbor, N.Y.

Hawiger J & Timmons S (1992) Binding of Fibrinogen and Von Willebrand Factor to Platelet Glycoprotein IIb-IIIa Complex. Methods Enzymol 215:228-243.

Hawiger J, Kloczewiak M, Bednarek M A & Timmons S (1989) Platelet Receptor Recognition Domains on the Alpha Chain of Human Fibrinogen: Structure-Function Analysis. Biochemistry 28:2909-2914.

Henikoff J G, Pietrokovski S, McCallum C M & Henikoff S (2000) Blocks-Based Methods for Detecting Protein Homology. Electrophoresis 21:1700-1706.

Henikoff S & Henikoff J G (1992) Amino Acid Substitution Matrices from Protein Blocks. Proc Natl Acad Sci U S A 89:10915-10919.

Henikoff S & Henikoff J G (2000) Amino Acid Substitution Matrices. Adv Protein Chem 54:73-97.

Hilmas D E & Gillette E L (1975) Microvasculature of C3h/Bi Mouse Mammary Tumors after X-lrradiation. Radiat Res 61:128-143.

Huang C C, Novak W R, Babbitt P C, Jewett A I, Ferrin T E & Klein T E (2000) Integrated Tools for Structural and Sequence Alignment and Analysis. Pac Symp Biocomput:230-241.

Ito T, Qiu H, Collins J A, Brill A B, Johnson D K & Griffin T W (1991) Preclinical Assessments of 90y-Labeled C110 Anti-Carcinoembryonic Antigen Immunotoxin: A Therapeutic Immunoconjugate for Human Colon Cancer. Cancer Res 51:255-260.

Janoff A S (1999) Liposomes: Rational Design. M. Dekker, New York.

Johnson R (1976) A Thermodynamic Method for Investigation of Radiation Induced Changes in the Microcirculation of Human Tumors. Int J Radiat Oncol Biol Phys 1:659-670.

Kallman R F, DeNardo G L & Stasch M J (1972) Blood Flow in Irradiated Mouse Sarcoma as Determined by the Clearance of Xenon-133. Cancer Res 32:483-490.

Karlin S & Altschul S F (1993) Applications and Statistics for Multiple High-Scoring Segments in Molecular Sequences. Proc Natl Acad Sci U S A 90:5873-5877.

Kendall R L, Wang G & Thomas K A (1996) Identification of a Natural Soluble Form of the Vascular Endothelial Growth Factor Receptor, Flt-1, and Its Heterodimerization with KDR. Biochem Biophys Res Commun 226:324-328.

Kendall R L, Wang G, DiSalvo J & Thomas K A (1994) Specificity of Vascular Endothelial Cell Growth Factor Receptor Ligand Binding Domains. Biochem Biophys Res Commun 201:326-330.

Kermani P, Leclerc G, Martel R & Fareh J (2001) Effect of Ionizing Radiation on Thymidine Uptake, Differentiation, and VEGFR2 Receptor Expression in Endothelial Cells: The Role of VEGF(165). Int J Radiat Oncol Biol Phys 50:213-220.

Kirpotin D, Park J W, Hong K, Zalipsky S, Li W L, Carter P, Benz C C & Papahadjopoulos D (1997) Sterically Stabilized Anti-Her2 Immunoliposomes: Design and Targeting to Human Breast Cancer Cells in Vitro. Biochemistry 36:66-75.

Kozin S V, Boucher Y, Hicklin D J, Bohlen P, Jain R K & Suit H D (2001) Vascular Endothelial Growth Factor Receptor-2-Blocking Antibody Potentiates Radiation-Induced Long-Term Control of Human Tumor Xenografts. Cancer Res 61:39-44.

Kruskal W & Wallis W (1952) Use of Ranks in One-Criteria Varinace Analysis. J Am Stat Assoc 47:583-621.

Kyte J & Doolittle R F (1982) A Simple Method for Displaying the Hydropathic Character of a Protein. J Mol Biol 157:105-132.

Labat-Moleur F, Steffan A M, Brisson C, Perron H, Feugeas O, Furstenberger P, Oberling F, Brambilla E & Behr J P (1996) An Electron Microscopy Study into the Mechanism of Gene Transfer with Lipopolyamines. Gene Ther3:1010-1017.

Lasic D D & Martin F J (1995) STEALTH® Liposomes. CRC Press, Boca Raton, Fla., United States of America.

Lee C G, Heijn M, di Tomaso E, Griffon-Etienne G, Ancukiewicz M, Koike C, Park K R, Ferrara N, Jain R K, Suit H D & Boucher Y (2000) Anti-Vascular Endothelial Growth Factor Treatment Augments Tumor Radiation Response under Normoxic or Hypoxic Conditions. Cancer Res 60:5565-5570.

Leibel S A & Phillips T L (1998) Textbook of Radiation Oncology. Saunders, Philadelphia, United States of America.

Leunig M, Yuan F, Menger M D, Boucher Y, Goetz A E, Messmer K & Jain R K (1992) Angiogenesis, Microvascular Architecture, Microhemodynamics, and Interstitial Fluid Pressure During Early Growth of Human Adenocarcinoma LS174T in SCID Mice. Cancer Res 52:6553-6560.

Lin P, Sankar S, Shan S, Dewhirst M W, Polverini P J, Quinn T Q & Peters K G (1998) Inhibition of Tumor Growth by Targeting Tumor Endothelium Using a Soluble Vascular Endothelial Growth Factor Receptor. Cell Growth Differ 9:49-58.

Lindegaard J C, Overgaard J, Bentzen S M & Pedersen D (1996) Is There a Radiobiologic Basis for Improving the Treatment of Advanced Stage Cervical Cancer? J Natl Cancer Inst Monogr 21:105-112.

Manome Y, Abe M, Hagen M F, Fine H A & Kufe D W (1994) Enhancer Sequences of the Df3 Gene Regulate Expression of the Herpes Simplex Virus Thymidine Kinase Gene and Confer Sensitivity of Human Breast Cancer Cells to Ganciclovir. Cancer Res 54:5408-5413.

Marin M, Noel D & Piechaczyk M (1997) Towards Efficient Cell Targeting by Recombinant Retroviruses. Mol Med Today 3:396-403.

Maruyama-Tabata H, Harada Y, Matsumura T, Satoh E, Cui F, Iwai M, Kita M, Hibi S, Imanishi J, Sawada T & Mazda O (2000) Effective Suicide Gene Therapy in Vivo by EBV-Based Plasmid Vector Coupled with Polyamidoamine Dendrimer. Gene Ther 7:53-60.

Millauer B, Wizigmann-Voos S, Schnurch H, Martinez R, Moller N P, Risau W & Ullrich A (1993) High Affinity VEGF Binding and Developmental Expression Suggest Flk-1 as a Major Regulator of Vasculogenesis and Angiogenesis. Cell 72:835-846.

Needleman S B & Wunsch C D (1970) A General Method Applicable to the Search for Similarities in the Amino Acid Sequence of Two Proteins. J Mol Biol 48:443-453.

Neri D, Carnemolla B, Nissim A, Leprini A, Querze G, Balza E, Pini A, Tarli L, Halin C, Neri P, Zardi L & Winter G (1997) Targeting by Affinity-Matured Recombinant Antibody Fragments of an Angiogenesis Associated Fibronectin Isoform. Nat Biotechnol 15:1271-1275.

Nor J E, Christensen J, Mooney D J & Polverini P J (1999) Vascular Endothelial Growth Factor (Vegf)-Mediated Angiogenesis Is Associated with Enhanced Endothelial Cell Survival and Induction of Bcl- 2 Expression. Am J Pathol 154:375-384.

Ohtsuka E, Matsuki S, Ikehara M, Takahashi Y & Matsubara K (1985) An Alternative Approach to Deoxyoligonucleotides as Hybridization Probes by Insertion of Deoxyinosine at Ambiguous Codon Positions. J Biol Chem 260:2605-2608.

Packer R J (1999) Brain Tumors in Children. Arch Neurol 56:421-425.

Park J W, Hong K, Kirpotin D B, Meyer O, Papahadjopoulos D & Benz C C (1997) Anti-Her2 Immunoliposomes for Targeted Therapy of Human Tumors. Cancer Lett 118:153-160.

Pasqualini R & Ruoslahti E (1996) Organ Targeting in Vivo Using Phage Display Peptide Libraries. Nature 380:364-366.

Pasqualini R, Koivunen E & Ruoslahti E (1997) Alpha V Integrins as Receptors for Tumor Targeting by Circulating Ligands. Nat Biotechnol 15:542-546.

Pearson W R & Lipman D J (1988) Improved Tools for Biological Sequence Comparison. Proc Natl Acad Sci U S A 85:2444-2448.

Rossolini G M, Cresti S, Ingianni A, Cattani P, Riccio M L & Satta G (1994) Use of Deoxyinosine-Containing Primers Vs Degenerate Primers for Polymerase Chain Reaction Based on Ambiguous Sequence Information. Mol Cell Probes 8:91-98.

Saltzman W M & Fung L K (1997) Polymeric Implants for Cancer Chemotherapy. Adv Drug Deliv Rev 26:209-230.

Sambrook J & Russell D W (2001) Molecular Cloning: A Laboratory Manual, 3rd ed. Cold Spring Harbor Laboratory Press, Cold Spring Harbor, N.Y.

Saqi M A, Wild D L & Hartshorn M J (1999) Protein Analyst—a Distributed Object Environment for Protein Sequence and Structure Analysis. Bioinformatics 15:521-522.

Shaheen R M, Davis D W, Liu W, Zebrowski B K, Wilson M R, Bucana C D, McConkey D J, McMahon G & Ellis L M (1999) Antiangiogenic Therapy Targeting the Tyrosine Kinase Receptor for Vascular Endothelial Growth Factor Receptor Inhibits the Growth of Colon Cancer Liver Metastasis and Induces Tumor and Endothelial Cell Apoptosis. Cancer Res 59:5412-5416.

Shintani S, Kiyota A, Mihara M, Nakahara Y, Terakado N, Ueyama Y & Matsumura T (2000) Association of Preoperative Radiation Effect with Tumor Angiogenesis and Vascular Endothelial Growth Factor in Oral Squamous Cell Carcinoma. Jpn J Cancer Res 91:1051-1057.

Silhavy T J, Berman M L, Enquist L W & Cold Spring Harbor Laboratory. (1984) Experiments with Gene Fusions. Cold Spring Harbor Laboratory, Cold Spring Harbor, N.Y.

Smith T F & Waterman M (1981) Comparison of Biosequences. Adv Appl Math 2:482-489.

Song C W, Payne J T & Levitt S H (1972) Vascularity and Blood Flow in X-Irradiated Walker Carcinoma 256 of Rats. Radiology 104:693-697.

Staba M J, Mauceri H J, Kufe D W, Hallahan D E & Weichselbaum R R (1998) Adenoviral TNF-Alpha Gene Therapy and Radiation Damage Tumor Vasculature in a Human Malignant Glioma Xenograft. Gene Ther 5:293-300.

Suit H (1996) Assessment of the Impact of Local Control on Clinical Outcome. Front Radiat Ther Oncol 29:17-23.

Taghian A, Ramsay J, Allalunis-Turner J, Budach W, Gioioso D, Pardo F, Okunieff P, Bleehen N, Urtasun R & Suit H (1993) Intrinsic Radiation Sensitivity May Not Be the Major Determinant of the Poor Clinical Outcome of Glioblastoma Multiforme. Int J Radiat Oncol Biol Phys 25:243-249.

Takayama K, Ueno H, Nakanishi Y, Sakamoto T, Inoue K, Shimizu K, Oohashi H & Hara N (2000) Suppression of Tumor Angiogenesis and Growth by Gene Transfer of a Soluble Form of Vascular Endothelial Growth Factor Receptor into a Remote Organ. Cancer Res 60:2169-2177.

Tam P, Monck M, Lee D, Ludkovski O, Leng E C, Clow K, Stark H, Scherrer P, Graham R W & Cullis P R (2000) Stabilized Plasmid-Lipid Particles for Systemic Gene Therapy. Gene Ther 7:1867-1874.

Tijssen (1993) Laboratory Techniques in Biochemistry and Molecular Bioloqy-Hybridization with Nucleic Acid Probes. Elsevier, N.Y.

Ting L L, Belfi C A, Tefft M & Ngo F Q (1991) KHT Sarcoma Blood Perfusion Change after Single-Dose X-Ray Irradiation. Int J Radiat Biol 60:335-339.

U.S. Pat. No. 4,196,265

U.S. Pat. No. 4,235,871

U.S. Pat. No. 4,551,482

U.S. Pat. No. 4,554,101

U.S. Pat. No. 5,011,634

U.S. Pat. No. 5,091,513

U.S. Pat. No. 5,111,867

U.S. Pat. No. 5,132,405

U.S. Pat. No. 5,234,933

U.S. Pat. No. 5,260,203

U.S. Pat. No. 5,326,902

U.S. Pat. No. 5,490,840

U.S. Pat. No. 5,510,103

U.S. Pat. No. 5,632,991

U.S. Pat. No. 5,651,991

U.S. Pat. No. 5,677,427

U.S. Pat. No. 5,667,988

U.S. Pat. No. 5,574,172

U.S. Pat. No. 5,688,931

U.S. Pat. No. 5,702,892

U.S. Pat. No. 5,714,166

U.S. Pat. No. 5,780,225

U.S. Pat. No. 5,786,387

U.S. Pat. No. 5,840,479

U.S. Pat. No. 5,849,877

U.S. Pat. No. 5,854,027

U.S. Pat. No. 5,855,900

U.S. Pat. No. 5,858,410

U.S. Pat. No. 5,892,019

U.S. Pat. No. 5,922,254

U.S. Pat. No. 5,922,356

U.S. Pat. No. 5,922,545

U.S. Pat. No. 5,948,647

U.S. Pat. No. 5,948,767

U.S. Pat. No. 5,985,279

U.S. Pat. No. 5,994,392

U.S. Pat. No. 6,054,561

U.S. Pat. No. 6,056,938

U.S. Pat. No. 6,057,098

U.S. Pat. No. 6,071,890

U.S. Pat. No. 6,090,925

U.S. Pat. No. 6,106,866

U.S. Pat. No. 6,120,787

U.S. Pat. No. 6,127,339

U.S. Pat. No. 6,132,766

U.S. Pat. No. 6,159,443

U.S. Pat. No. 6,174,708

U.S. Pat. No. 6,180,084

U.S. Pat. No. 6,190,700

U.S. Pat. No. 6,197,333

U.S. Pat. No. 6,200,598

U.S. Pat. No. 6,210,707

U.S. Pat. No. 6,217,886

U.S. Pat. No. 6,221,958

U.S. Pat. No. 6,238,704

U.S. Pat. No. 6,238,705

U.S. Pat. No. 6,245,740

U.S. Pat. No. 6,262,127

U.S. Pat. No. 6,267,981

U.S. Pat. No. 6,287,587

U.S. Pat. No. 6,296,832

U.S. Pat. No. 6,296,842

U.S. Pat. No. 6,312,713

U.S. Pat. No. 6,335,035

Vajkoczy P, Menger M D, Vollmar B, Schilling L, Schmiedek P, Hirth K P, Ulirich A & Fong T A (1999) Inhibition of Tumor Growth, Angiogenesis, and Microcirculation by the Novel Flk-1 Inhibitor SU5416 as Assessed by Intravital Multi-Fluorescence Videomicroscopy. Neoplasia 1:31-41.

Valter M M, Hugel A, Huang H J, Cavenee W K, Wiestler O D, Pietsch T & Wernert N (1999) Expression of the Ets-1 Transcription Factor in Human Astrocytomas Is Associated with Fms-Like Tyrosine Kinase-1 (Flt-1)/Vascular Endothelial Growth Factor Receptor-1 Synthesis and Neoangiogenesis. Cancer Res 59:5608-5614.

Walker M D, Green S B, Byar D P, Alexander E, Jr., Batzdorf U, Brooks W H, Hunt W E, MacCarty C S, Mahaley M S, Jr., Mealey J, Jr., Owens G, Ransohoff J, 2nd, Robertson J T, Shapiro W R, Smith K R, Jr., Wilson C B & Strike T A (1980) Randomized Comparisons of Radiotherapy and Nitrosoureas for the Treatment of Malignant Glioma after Surgery. N Engl J Med 303:1323-1329.

Wallner K E, Galicich J H, Krol G, Arbit E & Malkin M G (1989) Patterns of Failure Following Treatment for Glioblastoma Multiforme and Anaplastic Astrocytoma. Int J Radiat Oncol Biol Phys 16:1405-1409.

Waltenberger J, Claesson-Welsh L, Siegbahn A, Shibuya M & Heldin C H (1994) Different Signal Transduction Properties of KDR and Flt1, Two Receptors for Vascular Endothelial Growth Factor. J Biol Chem 269:26988-26995.

WO 93/25521

WO 98/10795

Yamaura H, Yamada K & Matsuzawa T (1976) Radiation Effect on the Proliferating Capillaries in Rat Transparent Chambers. Int J Radiat Biol Relat Stud Phys Chem Med 30:179-187.

It will be understood that various details of the invention can be changed without departing from the scope of the invention. Furthermore, the foregoing description is for the purpose of illustration only, and not for the purpose of limitation—the invention being defined by the claims appended hereto.

What is claimed is:

1. A method for increasing radiosensitivity of a tumor in a subject comprising administering a soluble VEGF-R polypeptide to a tumor in a subject, whereby the radiosensitivity of the tumor is increased.

2. The method of claim 1, wherein the administering comprises administering a composition comprising:
(a) a soluble VEGF-R polypeptide; and
(b) a pharmaceutically acceptable carrier.

3. The method of claim 2, wherein the pharmaceutically acceptable carrier comprises a gene therapy construct that encodes the soluble VEGF-R polypeptide.

4. The method of claim 1, wherein the administering comprises administering a minimally therapeutic dose of a soluble VEGF-R polypeptide.

5. The method of claim 1, wherein the soluble VEGF-R polypeptide comprises:
(a) a polypeptide encoded by a nucleic acid of SEQ ID NO: 1 or 3;
(b) a polypeptide encoded by a nucleic acid substantially identical to SEQ ID NO: 1 or 3;
(c) a polypeptide comprising an amino acid sequence of SEQ ID NO: 2 or 4; or
(d) a polypeptide substantially identical to SEQ ID NO: 2 or 4.

6. The method of claim 1, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 1 or 3 under wash stringency conditions represented by a wash solution having less than about 200 mM salt concentration and a wash temperature of greater than about 45° C., and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

7. The method of claim 1, wherein the soluble VEGF-R polypeptide comprises:
(a) a polypeptide encoded by a nucleic acid of SEQ ID NO: 5 or 7;
(b) a polypeptide encoded by a nucleic acid substantially identical to SEQ ID NO: 5 or 7;
(c) a polypeptide comprising an amino acid sequence of SEQ ID NO: 6 or 8; or
(d) a polypeptide substantially identical to SEQ ID NO: 6 or 8.

8. The method of claim 1, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 5 or 7 under wash stringency conditions represented by a wash solution having less than about 200 mM salt concentration and a wash temperature of greater than about 45° C.; and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

9. The method of claim 1, wherein the tumor comprises a radiation resistant tumor.

10. The method of claim 1, wherein the subject comprises a mammal.

11. The method of claim 10, wherein the mammal comprises a human.

12. A method for delaying tumor growth, the method comprising:
(a) administering a VEGF-R2 inhibitor to a subject bearing a tumor; whereby the radiosensitivity of the tumor is increased; and
(b) treating the tumor with ionizing radiation, whereby tumor growth is delayed.

13. The method of claim 12, wherein the administering a VEGF-R2 inhibitor comprises administering a composition comprising:
(a) a VEGF-R2 inhibitor; and
(b) a pharmaceutically acceptable carrier.

14. The method of claim 13, wherein the VEGF-R2 inhibitor comprises SU5416.

15. The method of claim 13, wherein the VEGF-R2 inhibitor comprises an antibody that specifically binds to VEGF-R2.

16. The method of claim 13, wherein the VEGF-R2 inhibitor comprises a soluble VEGF-R polypeptide.

17. The method of claim 16, wherein the soluble VEGF-R polypeptide comprises:
(a) a polypeptide encoded by a nucleic acid of SEQ ID NO: 1 or 3;
(b) a polypeptide encoded by a nucleic acid substantially identical to SEQ ID NO: 1 or 3;
(c) a polypeptide comprising an amino acid sequence of SEQ ID NO: 2 or 4; or
(d) a polypeptide substantially identical to SEQ ID NO: 2 or 4.

18. The method of claim 16, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 1 or 3 under wash stringency conditions represented by a wash solution having less than about 200 mM salt concentration and a wash temperature of greater than about 45° C., and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

19. The method of claim 16, wherein the soluble VEGF-R polypeptide comprises:
(a) a polypeptide encoded by a nucleic acid of SEQ ID NO: 5 or 7;
(b) a polypeptide encoded by a nucleic acid substantially identical to SEQ ID NO: 5 or 7;
(c) a polypeptide comprising an amino acid sequence of SEQ ID NO: 6 or 8; or
(d) a polypeptide substantially identical to SEQ ID NO: 6 or 8.

20. The method of claim 16, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 5 or 7 under wash stringency conditions represented by a wash solution having less than about 200 mM salt concentration and a wash temperature of greater than about 45° C., and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

21. The method of claim 13, wherein the pharmaceutically acceptable carrier comprises a gene therapy vector.

22. The method of claim 12, wherein the administering a VEGF-R2 inhibitor comprises administering a minimally therapeutic dose of a VEGF-R2 inhibitor.

23. The method of claim 12, wherein the tumor comprises a radiation resistant tumor.

24. The method of claim 12, wherein the subject comprises a mammal.

25. The method of claim 24, wherein the mammal comprises a human.

26. The method of claim 12, wherein the treating the tumor with ionizing radiation comprises treating the tumor with a subtherapeutic dose of ionizing radiation.

27. The method of claim 12, further comprising promoting tumor regression.

28. A method for inhibiting tumor blood vessel growth, the method comprising:
(a) administering a VEGF-R2 inhibitor to a subject bearing a tumor; whereby the radiosensitivity of the tumor is increased; and
(b) treating the tumor with ionizing radiation, whereby tumor blood vessel growth is inhibited.

29. The method of claim 28, wherein the administering a VEGF-R2 inhibitor comprises administering a composition comprising:
(a) a VEGF-R2 inhibitor; and
(b) a pharmaceutically acceptable carrier.

30. The method of claim 29, wherein the VEGF-R2 inhibitor comprises SU5416.

31. The method of claim 29, wherein the VEGF-R2 inhibitor comprises an antibody that specifically binds to VEGF-R2.

32. The method of claim 29, wherein the VEGF-R2 inhibitor comprises a soluble VEGF-R polypeptide.

33. The method of claim 32, wherein the soluble VEGF-R polypeptide comprises:
(a) a polypeptide encoded by a nucleic acid of SEQ ID NO: 1 or 3;
(b) a polypeptide encoded by a nucleic acid substantially identical to SEQ ID NO: 1 or 3;
(c) a polypeptide comprising an amino acid sequence of SEQ ID NO: 2 or 4; or
(d) a polypeptide substantially identical to SEQ ID NO: 2 or 4.

34. The method of claim 32, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 1 or 3 under wash stringency conditions represented by a wash solution having less than about 200 mM salt concentration and a wash temperature of greater than about 45° C., and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

35. The method of claim 32, wherein the soluble VEGF-R polypeptide comprises:
(a) a polypeptide encoded by a nucleic acid of SEQ ID NO: 5 or 7;
(b) a polypeptide encoded by a nucleic acid substantially identical to SEQ ID NO: 5 or 7;
(c) a polypeptide comprising an amino acid sequence of SEQ ID NO: 6 or 8; or
(d) a polypeptide substantially identical to SEQ ID NO: 6 or 8.

36. The method of claim 32, wherein the soluble VEGF-R polypeptide comprises a polypeptide encoded by an isolated nucleic acid segment selected from the group consisting of:
(a) an isolated nucleic acid molecule which hybridizes to a nucleic acid sequence of SEQ ID NO: 5 or 7 under wash stringency conditions represented by a wash solution having less than about 200 mM salt concentration and a wash temperature of greater than about 45° C., and which encodes a VEGF-R polypeptide; and
(b) an isolated nucleic acid molecule differing by at least one functionally equivalent codon from the isolated nucleic acid molecule of (a) above in nucleic acid sequence due to the degeneracy of the genetic code, and which encodes a VEGF-R polypeptide encoded by the isolated nucleic acid of (a) above.

37. The method of claim 28, wherein the pharmaceutically acceptable carrier comprises a gene therapy construct that encodes the VEGF-R2 inhibitor.

38. The method of claim 28, wherein the administering a VEGF-R2 inhibitor comprises administering a minimally therapeutic dose of a VEGF-R2 inhibitor.

39. The method of claim 28, wherein the tumor comprises a radiation resistant tumor.

40. The method of claim 28, wherein the subject comprises a mammal.

41. The method of claim 40, wherein the mammal comprises a human.

42. The method of claim 28, wherein the treating the tumor with ionizing radiation comprises treating the tumor with a subtherapeutic dose of ionizing radiation.

43. The method of claim 28, further comprising delaying tumor growth.

44. The method of claim 43, further comprising promoting tumor regression.


SEQUENCE LISTING (8 sequences)

SEQ ID NO: 1 - DNA, 2271 bp, Mus musculus
  CDS (79)..(2271)
gacttctttgcgggccagggacggagaaggagtctgtgcctgagaaactgggctctgtgcccaggcgcgaggtgcaggatggagagcaaggcgctgctagctgtcgctctgtggttctgcgtggagacccgagccgcctctgtgggtttgactggcgattttctccatccccccaagctcagcacacagaaagacatactgacaattttggcaaatacaacccttcagattacttgcaggggacagcgggacctggactggctttggcccaatgctcagcgtgattctgaggaaagggtattggtgactgaatgcggcggtggtgacagtatcttctgcaaaacactcaccattcccagggtggttggaaatgatactggagcctacaagtgctcgtaccgggacgtcgacatagcctccactgtttatgtctatgttcgagattacagatcaccattcatcgcctctgtcagtgaccagcatggcatcgtgtacatcaccgagaacaagaacaaaactgtggtgatcccctgccgagggtcgatttcaaacctcaatgtgtctctttgcgctaggtatccagaaaagagatttgttccggatggaaacagaatttcctgggacagcgagataggctttactctccccagttacatgatcagctatgccggcatggtcttctgtgaggcaaagatcaatgatgaaacctatcagtctatcatgtacatagttgtggttgtaggatataggatttatgatgtgattctgagccccccgcatgaaattgagctatctgccggagaaaaacttgtcttaaattgtacagcgagaacagagctcaatgtggggcttgatttcacctggcactctccaccttcaaagtctcatcataagaagattgtaaaccgggatgtgaaaccctttcctgggactgtggcgaagatgtttttgagcaccttgacaatagaaagtgtgaccaagagtgaccaaggggaatacacctgtgtagcgtccagtggacggatgatcaagagaaatagaacatttgtccgagttcacacaaagccttttattgctttcggtagtgggatgaaatctttggtggaagccacagtgggcagtcaagtccgaatccctgtgaagtatctcagttacccagctcctgatatcaaatggtacagaaatggaaggcccattgagtccaactacacaatgattgttggcgatgaactcaccatcatggaagtgactgaaagagatgcaggaaactacacggtcatcctcaccaaccccatttcaatggagaaacagagccacatggtctctctggttgtgaatgtcccaccccagatcggtgagaaagccttgatctcgcctatggattcctaccagtatgggaccatgcagacattgacatgcacagtctacgccaaccctcccctgcaccacatccagtggtactggcagctagaagaagcctgctcctacagacccggccaaacaagcccgtatgcttgtaaagaatggagacacgtggaggatttccaggggggaaacaagatcgaagtcaccaaaaaccaatatgccctgattgaaggaaaaaacaaaactgtaagtacgctggtcatccaagctgccaacgtgtcagcgttgtacaaatgtgaagccatcaacaaagcgggacgaggagagagggtcatctccttccatgtgatcaggggtcctgaaattactgtgcaacctgctgcccagccaactgagcaggagagtgtgtccctgttgtgcactgcagacagaaatacgtttgagaacctcacgtggtacaagcttggctcacaggcaacatcggtccacatgggcgaatcactcacaccagtttgcaagaacttggatgctctttggaaactgaatggcaccatgttttctaacagcacaaatgacatcttgattgtggcatttcagaatgcctctctgcaggaccaaggcgactatgtttgctctgctcaagataagaagaccaagaaaagacattgcctggtcaaacagctcatcatcctagagcgcatggcacccatgatcaccggaaatctggagaatcagacaacaaccattggcgagaccattgaagtgacttgcccagcatctggaaatcctaccccacacattacatggttcaaagacaacgagaccctggtagaagattcaggcattgtactgagagatgggaaccggaacctgactatccgcagggtgaggaaggaggatggaggc

SEQ ID NO: 2 - protein, 731 aa, Mus musculus
MESKALLAVALWFCVETRAASVGLTGDFLHPPKLSTQKDILTILANTTLQITCRGQRDLDWLWPNAQRDSEERVLVTECGGGDSIFCKTLTIPRVVGNDTGAYKCSYRDVDIASTVYVYVRDYRSPFIASVSDQHGIVYITENKNKTVVIPCRGSISNLNVSLCARYPEKRFVPDGNRISWDSEIGFTLPSYMISYAGMVFCEAKINDETYQSIMYIVVVVGYRIYDVILSPPHEIELSAGEKLVLNCTARTELNVGLDFTWHSPPSKSHHKKIVNRDVKPFPGTVAKMFLSTLTIESVTKSDQGEYTCVASSGRMIKRNRTFVRVHTKPFIAFGSGMKSLVEATVGSQVRIPVKYLSYPAPDIKWYRNGRPIESNYTMIVGDELTIMEVTERDAGNYTVILTNPISMEKQSHMVSLVVNVPPQIGEKALISPMDSYQYGTMQTLTCTVYANPPLHHIQWYWQLEEACSYRPGQTSPYACKEWRHVEDFQGGNKIEVTKNQYALIEGKNKTVSTLVIQAANVSALYKCEAINKAGRGERVISFHVIRGPEITVQPAAQPTEQESVSLLCTADRNTFENLTWYKLGSQATSVHMGESLTPVCKNLDALWKLNGTMFSNSTNDILIVAFQNASLQDQGDYVCSAQDKKTKKRHCLVKQLIILERMAPMITGNLENQTTTIGETIEVTCPASGNPTPHITWFKDNETLVEDSGIVLRDGNRNLTIRRVRKEDGG

SEQ ID NO: 3 - DNA, 2320 bp, Homo sapiens
  CDS (1)..(2319)
atgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtga

SEQ ID NO: 4 - protein, 773 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAV

SEQ ID NO: 5 - DNA, 2067 bp, Mus musculus
  CDS (1)..(2067)
atggtcagctgctgggacaccgcggtcttgccttacgcgctgctcgggtgtctgcttctcacaggatatggctcagggtcgaagttaaaagtgcctgaactgagtttaaaaggcacccagcatgtcatgcaagcaggccagactctctttctcaagtgcagaggggaggcagcccactcatggtctctgcccacgaccgtgagccaggaggacaaaaggctgagcatcactcccccatcggcctgtgggagggataacaggcaattctgcagcaccttgaccttggacacggcgcaggccaaccacacgggcctctacacctgtagatacctccctacatctacttcgaagaaaaagaaagcggaatcttcaatctacatatttgttagtgatgcagggagtcctttcatagagatgcacactgacatacccaaacttgtgcacatgacggaaggaagacagctcatcatcccctgccgggtgacgtcacccaacgtcacagtcaccctaaaaaagtttccatttgatactcttacccctgatgggcaaagaataacatgggacagtaggagaggctttataatagcaaatgcaacgtacaaagagataggactgctgaactgcgaagccaccgtcaacgggcacctgtaccagacaaactatctgacccatcggcagaccaatacaatcctagatgtccaaatacgcccgccgagcccagtgagactgctccacgggcagactcttgtcctcaactgcaccgccaccacggagctcaatacgagggtgcaaatgagctggaattaccctggtaaagcaactaagagagcatctataaggcagcggattgaccggagccattcccacaacaatgtgttccacagtgttcttaagatcaacaatgtggagagccgagacaaggggctctacacctgtcgcgtgaagagtgggtcctcgttccagtctttcaacacctccgtgcatgtgtatgaaaaaggattcatcagtgtgaaacatcggaagcagccggtgcaggaaaccacagcaggaagacggtcctatcggctgtccatgaaagtgaaggccttcccctccccagaaatcgtatggttaaaagatggctcgcctgcaacattgaagtctgctcgctatttggtacatggctactcattaattatcaaagatgtgacaaccgaggatgcaggggactatacgatcttgctgggcataaagcagtcaaggctatttaaaaacctcactgccactctcattgtaaacgtgaaacctcagatctacgaaaagtccgtgtcctcgcttccaagcccacctctctatccgctgggcagcagacaagtcctcacttgcaccgtgtatggcatccctcggccaacaatcacgtggctctggcacccctgtcaccacaatcactccaaagaaaggtatgacttctgcactgagaatgaagaatcctttatcctggatcccagcagcaacttaggaaacagaattgagagcatctctcagcgcatgacggtcatagaaggaacaaataagacggttagcacattggtggtggctgactctcagacccctggaatctacagctgccgggccttcaataaaatagggactgtggaaagaaacataaaattttacgtcacagatgtgccgaatggctttcacgtttccttggaaaagatgccagccgaaggagaggacctgaaactgtcctgtgtggtcaataaattcctgtacagagacattacctggattctgctacggacagttaacaacagaaccatgcaccatagtatcagcaagcaaaaaatggccaccactcaagattactccatcactctgaaccttgtcatcaagaacgtgtctctagaagactcgggcacctatgcgtgcagagccaggaacatatacacaggggaagacatccttcggaagacagaagttctcgttagaggtgagcactgcggcaaaaaggccattttctctcggatctccaaatttaaaagcaggaggaatgattgtaccacacaaagtcatgtcaaacattaa

SEQ ID NO: 6 - protein, 688 aa, Mus musculus
MVSCWDTAVLPYALLGCLLLTGYGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKGFISVKHRKQPVQETTAGRRSYRLSMKVKAFPSPEIVWLKDGSPATLKSARYLVHGYSLIIKDVTTEDAGDYTILLGIKQSRLFKNLTATLIVNVKPQIYEKSVSSLPSPPLYPLGSRQVLTCTVYGIPRPTITWLWHPCHHNHSKERYDFCTENEESFILDPSSNLGNRIESISQRMTVIEGTNKTVSTLVVADSQTPGIYSCRAFNKIGTVERNIKFYVTDVPNGFHVSLEKMPAEGEDLKLSCVVNKFLYRDITWILLRTVNNRTMHHSISKQKMATTQDYSITLNLVIKNVSLEDSGTYACRARNIYTGEDILRKTEVLVRGEHCGKKAIFSRISKFKSRRNDCTTQSHVKH

SEQ ID NO: 7 - DNA, 2061 bp, Homo sapiens
  CDS (1)..(2061)
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtc

SEQ ID NO: 8 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGV